(19) United States
(12) Patent Application Publication
(10) Pub. No.: US 2022/0370482 A1
(43) Pub. Date: Nov. 24, 2022

(54) INHIBITOR OF BINDING BETWEEN PODOCIN AND KERATIN 8 FOR USE IN THE TREATMENT OF NEPHROTIC SYNDROME

(71) Applicant: THE UNIVERSITY OF BRISTOL, Bristol (GB)

(72) Inventors: Moin Ahson SALEEM-UDDIN, Bristol (GB); Gavin Ian WELSH, Bristol (GB)

(73) Assignee: THE UNIVERSITY OF BRISTOL, Bristol (GB)

(21) Appl. No.: 17/774,051

(22) PCT Filed: Nov. 5, 2020
(86) PCT No.: PCT/EP2020/081114

§ 371 Date: May 3, 2022

(30) Foreign Application Priority Data
Nov. 5, 2019 (GB) 1916071.2

(51) Int. Cl. A61K 31/663; A61K 31/662; A61P 13/12

(52) U.S. Cl. CPC A61K 31/663; A61K 31/662; A61P 13/12

(57) ABSTRACT

A compound for use in the treatment of nephrotic syndrome in a subject in need thereof, wherein the compound is an inhibitor of binding between a target protein and keratin 8.

### TECHNICAL FIELD

The invention relates to a compound for use in the treatment of nephrotic syndrome, particularly a compound which is an inhibitor of binding between a target protein and keratin 8. The invention also relates to a kit comprising the compound, a method of treatment using the compound, and use of the compound in a method of manufacturing a medicament.

### BACKGROUND

Nephrotic syndrome (NS) is described as a triad of heavy proteinuria (>40 mg/m²/hr), oedema and hypoalbuminaemia (<3.0 g/dL), and often results in end-stage renal disease (ESRD), accounting for 15% of the cases in the European population. Even though NS is linked to various types of renal disease, the most common form (90%) identified in children is idiopathic NS, which progresses in the absence of any clinical features of primary extrarenal disorder (T.-S. Ha, Korean J. Pediatr., vol. 60, no. 3, p. 55, 2017).

Clinically, NS can be classified into two groups by the response to steroid therapy: steroid-sensitive NS (SSNS) and steroid-resistant NS (SRNS). Most patients with idiopathic NS initially respond well to steroids and enter the remission stage with a good renal prognosis; however, around 90% of these patients will relapse, with another half of those becoming steroid-dependent. The remaining 10% are recognized to have SRNS. However, this fails to explain why some patients who originally respond to steroid treatment later become resistant. NS is considered to be the most prevalent glomerular disease of childhood, with an incidence of around 2 in 100,000 children. Approximately 20% of children will be steroid resistant, with a further 60% of these cases presenting with focal segmental glomerulosclerosis (FSGS), indicated by biopsy (M. A. Saleem, Pediatr. Nephrol., vol. 28, no. 5, pp. 699-709, May 2013.). FSGS is described as scarring of the glomerulus that includes several distinct changes, where only a segment of the glomerulus and some, but not all, glomeruli are affected. Progression to ESRD is closely correlated with the development of FSGS. Therefore, because most cases of genetic NS are clinically steroid-resistant with pathologically prevalent FSGS, genetic NS is hard to treat, has poor renal prognosis and often results in ESRD.

The majority of the SRNS genetic forms show structural alterations in the glomerulus or, more precisely, the podocyte. Molecular research work on the genetics of hereditary NS has revealed the podocyte as a key player in regulating glomerular filtration, whose structure and function are essential in the maintenance of the slit diaphragm membrane. To date, around 75 genes including those encoding nephrin, transient receptor potential canonical channel-6 (TRPC6), CD2AP, α-actinin-4 and podocin, have been reported to cause NS, and yet more remain to be identified. The significance of several of them, including nephrin, CD2AP and podocin, which participate in the slit diaphragm assembly, has been shown by the presence of heavy proteinuria, when they are mutated (M.-C. Gubler, J. Am. Soc. Nephrol., vol. 14, no. 90001, p. 22S-26, Jun. 2003).

One such protein, podocin, is the protein product of the NPHS2 gene that is mutated in a subset of patients with autosomal recessive SRNS, which manifests as early childhood onset of proteinuria, fast progression to ESRN and FSGS. Podocin is a novel 42 kDa podocyte specific integral membrane protein, and is a member of the stomatin family of proteins. In podocytes, podocin is exclusively localized to the slit diaphragm, where it is involved in mechanotransduction events (T. B. Huber et al., J. Biol. Chem., vol. 276, no. 45, pp. 41543— 6, Nov. 2001). This crucial localization of podocin to the slit diaphragm is underscored by the fact that mice lacking podocin present with massive proteinuria, foot process effacement and absence of slit diaphragms. Multiple experimental studies show that podocin in its oligomeric form localizes to lipid raft microdomains in the slit diaphragm, where it recruits and colocalizes with nephrin. As mentioned previously, the slit diaphragm of podocytes is essential for the correct function of the GFB and is assembled in lipid rafts.

Paediatric patients carrying truncating mutations of podocin (nonsense or frameshift), or homozygous p.R138Q, present with a severe, early-onset from of SRNS, which is found in 98.2% of cases studied (B. Hinkes et al., J. Am. Soc. Nephrol., vol. 19, no. 2, pp. 365-71, Feb. 2008). The replacement of the arginine residue at position 138, which is highly conserved amongst the stomatin-like family of proteins and is crucial for podocin function, with glutamine results in podocin with the R138Q mutation (N. Boute et al., Nat. Genet., vol. 24, no. 4, pp. 349-54, Apr. 2000). Functional data have shown that this protein is retained in the endoplasmic reticulum (ER) and loses its ability to target nephrin to lipid raft microdomains, therefore augmenting its signaling (T. B. Huber et al., Hum. Mol. Genet., vol. 12, no. 24, pp. 3397-405, December 2003.

As set out above, genetic NS is hard to treat, has poor renal prognosis and often results in ESRD. There is therefore a significant unmet need in provision of a therapeutic for treatment of NS.

### SUMMARY OF THE INVENTION

The present invention provides a compound for use in the treatment of nephrotic syndrome in a subject in need thereof, wherein the compound is an inhibitor of binding between a target protein and keratin 8.

The present invention also provides a compound for use in the treatment of nephrotic syndrome in a subject in need thereof, wherein the compound is a compound of formula I, or a pharmaceutically acceptable salt thereof:

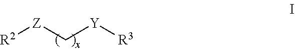

wherein x is 0 or an integer from 1 to 6;

Y is selected from the group consisting of: direct bond, C(O), C(O)O, 0, C(R¹)(OH), C(O)NR¹, S(O), S(O)(O) and P(O)(OH),

Z is selected from the group consisting of: C(0), C(O)O, 0, C(R′)(OH), C(O)NR^{I}, S(0), S(0)(0) and P(0)(OR¹);

wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;

R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl; and

R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

The present invention also provides a compound for use in the treatment of nephrotic syndrome in a subject in need thereof, wherein the compound is a compound of formula II, or a pharmaceutically acceptable salt thereof:

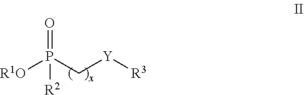

wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;

R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;

x is 0 or an integer from 1 to 6;

Y is selected from the group consisting of: direct bond, C(0), S(0), C(O)O, C(O)NH, and P(O)(OH); and

R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

The present invention also provides a kit comprising a compound which is an inhibitor of binding between a target protein and keratin 8 together with instructions for treating nephrotic syndrome.

The present invention also provides a method of treating nephrotic syndrome in a subject in need thereof comprising administering to said subject an effective amount of a compound which is an inhibitor of binding between a target protein and keratin 8.

The present invention also provides the use of a compound which is an inhibitor of binding between a target protein and keratin 8 in the manufacture of a medicament for the treatment of nephrotic syndrome in a subject in need thereof.

### BRIEF DESCRIPTION OF THE DRAWINGS

One or more embodiments of the invention will now be described, by way of example only, with reference to the accompanying drawings, in which:

FIG. 1 shows the effect of disease-causing podocin mutation: (a) Confocal images showing immunofluorescence staining of GFP-tagged podocin constructs and the ER protein calnexin in conditionally immortalized human podocytes. Scale bar=1 μm. (b) TIRF images of GFP-tagged podocin constructs and an early endosomal marker-1 (EEA1) with F-actin. Scale bar=0.75 μm. (c) Lipid raft isolation experiment of either the endogenous wild-type or mutant podocins. The quality and purity of the fractions were examined using Flotillin-1, as a lipid raft marker, and absence of CD99, as a plasma membrane marker.

FIG. 2 shows analysis of Keratin 8/18 expression levels using Immunofluorescence, Western Blotting and qPCR. (a, b) Colocalization of K8/K18 and GFP-tagged podocins in human podocytes. Scale bar=1 rim. (c, c′) Immunoblot analysis showing that K8 and K18 protein expression levels are significantly increased in mutant podocytes compared to wild-type cells. Differentiated ciPod n=3, two-tailed T-test (d, d′) RNA expression profiles of keratin 8 and keratin 18, two-tailed T-test*0.05

FIG. 3 shows keratin 8-R138Q podocin interaction: (a) Anti-keratin 8 antibody precipitated keratin 8 and co-precipitated podocin in human podocytes derived from the patient with R138Q mutation. Mouse-IgG was used as a control. (b) Molecular proximity of K8 and wild-type/R138Q podocin in human podocytes. No primary antibody was used as a negative control. Scale bar=20 μm. (b′) Results are representative of 3 independent experiments, two-tailed T-test

FIG. 4 shows effects of Keratin 8 silencing: (a) Western blots of protein lysates showing the effect of shRNA treatment on keratin 8 expression in human podocytes derived either from a healthy patient or a patient with the R138Q mutation. Cells were treated either with non-coding shRNA, GAPDH shRNA or with Seq. 1/2/3 of K8 KO shRNA. (b, b′) Seq.1 of K8 KO shRNA restored the association of R138Q podocin with lipid rats. The quality and purity of the fractions were examined using Flotillin-1 as lipid raft marker and the exclusion of CD99 as a marker for non-raft associated proteins. Two-tailed T-test (c) The adhesive defect of the cells was rescued using K8 shRNA sequence, while GAPDH shRNA had no effect. One-way ANOVA p≤0.05.

FIG. 5 shows treatment with compound Ia rescues R138Q podocin localization. (a) Podocytes stably expressing either WT podocin or R138Q podocin mutant were subjected to GFP and K8 immunodetection and analysed by confocal microscopy. (b) Effect of compound Ia treatment on the K8/18 protein expression levels in human podocytes derived from either a healthy patient or a patient with R138Q mutation. Results are representative of 3 independent experiments, two-tailed T-test (c) Co-IP was performed to see whether treatment with compound Ia disrupts K8-R138Q podocin interaction in human mutant podocytes.

FIG. 6 shows effect of compound Ia on the R138Q podocin's function and interaction: (a) Proximity ligation assay of K8 and WT/R138Q podocin in human podocytes derived either from a healthy patient or a patient with the R138Q mutation. Green spots correspond to K8/Podocin interaction (proximity of less than 40 nm). Nuclei (DAPI) is stained blue. Results are representative of at least three independent experiments; one-way ANOVA p<0.05. (b) R138Q podocin is found in the DRM fraction (fraction 1-4) after compound Ia treatment. Flotillin-1, raft marker, was used to confirm the purity of the fractions. Two-tailed T-test *<0.05. (c) Cell adhesion assay to see the effect of compound Ia on the adhesion of both cell types. Differentiated ciPod n=3, Two-tailed T-test*<0.05.

FIG. 7 shows the effects of in vivo treatment with compound Ia (a) Induced control mice developed severe proteinuria at 4 weeks after doxycycline induction, which was prevented in compound Ia mice. One-way ANOVA r:10.05. (b) Control mice display hypercholesterolemia, hypoalbuminemia and high blood urea levels, all of which is prevented upon administration of compound Ia. One-way ANOVA p≤0.05. (c) Treatment with compound Ia prevented podocyte loss in R140Q mice. One-way ANOVA p≤0.05. (d) compound Ia treatment had no effect on the proteinuria levels of the NPHS2^{flox/flox} mice.

FIG. 8 shows immunofluorescence and histological analysis: (a) Confocal images of podocin and nephrin performed on the NPHS2^{flox/R140Q} mice kidney sections. Treatment with compound Ia rescued the mutant podocin localization in mice. Magnification, X63; zoom, x8. (b) Keratin 8 expression was elevated in glomeruli of diseased animals, which was brought back to normal upon treatment with compound Ia, as seen by immunofluorescence. Magnification, X63; zoom, x8. (c) These mice developed FSGS with the large percentage of glomeruli affected by sclerosis. Mice that were given compound Ia demonstrated normal histology. Magnification, x40. Podocytes loss and effacement was observed in doxy+saline mice. Scale bar=500 nm.

FIG. 9 shows the results of screening compounds IIb-h in the cell adhesion assay.

FIG. 10 shows the NPHS2 floxed allele, where mice were created to carry a floxed NPHS2 exon 2 alleles, which can be excised using Cre recombinase.

FIG. 11 shows an example of breeding strategy used to generate a Conditional Knock-In Mouse Model of the R140Q Mutation. Only male mice had TetO-Cre and were used for further breeding, as TetO-Cre was shown to be leaky in females.

FIG. 12 shows the results of a high-throughput screening assay conducted using compound 407 are shown in FIG. 12: (a) shows a podocin R138Q mutant (PM) GlomSphere where podocytes (fluorescence) are shown to incompletely cover the GlomSphere surface. (b) shows untreated WT (wild type) GlomSphere where podocytes show much greater coverage of the GlomSphere surface, increasing its apparent size. (c) shows a podocin mutant spheroid treated for 5 days with compound 407. Podocyte retention has been improved and the GlomSphere looks more similar to the wild type condition. (d) shows quantified GFP mean fluorescence intensity of GlomSpheres made with podocin mutant podocytes, treated with compound 407. The compound can be seen to restore GFP fluorescence (and therefore podocyte retention) to closer to wildtype (WT) levels when compared to untreated GlomSpheres (PM). (e) shows the same experiment as (d), but with the GFP fluorescence adjusted for GlomSphere size (quantified integrated density of GlomSphere).

FIG. 13 shows formation and reorganization of glomerular spheroids. (a) shows the sequence of spheroid formation. A core of glomerular endothelial cells (GENCs) is first formed from 5,000 cells. A peripheral coating of podocytes is then wrapped around the GEnC core, forming a spheroid with a distinct boundary between the two cell types. (b) shows differentiation of a glomerular spheroid over 10 days. Peripheral coating of podocytes is shown to migrate around the GEnC core. Overall spheroid diameter is reduced from—380 μm (day 1) to ˜220 μm (day 10).

FIG. 14 shows immunohistochemistry stain for Keratin 8 on human glomerulus biopsy. Left panel is a biopsy from normal human glomerulus, with no expression in glomerulus. Right panel, biopsy from a patient with a R138Q podocin mutation. In the right panel, staining indicates Keratin 8 expression in cells consistent with a podocyte distribution. Proximal tubular expression is also noted.

### DETAILED DESCRIPTION

The term “pharmaceutically acceptable salt” used herein refers to a salt of the compounds described herein which is generally safe, non-toxic and neither biologically nor otherwise undesirable and includes that which is suitable for veterinary use as well as human pharmaceutical use. Examples of pharmaceutically acceptable salts are discussed in Berge et al (J. Pharm. Sci., 1977, 66, 1-19). Pharmaceutically acceptable salt forms include pharmaceutically acceptable acidic/anionic or basic/cationic salts.

Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, methanesulfonate and p-toluenesulfonate and triethiodide salts. Examples of the alkali addition salts include inorganic salts such as, for example, sodium, potassium, calcium and magnesium, and organic alkali salts such as, for example, ethylenediamine, ethanolamine, N,N-dialkylenethanolamine, triethanolamine and basic amino acids salts. Examples of suitable organic cations include ammonium ion (i.e., NH⁴⁺) and substituted ammonium ions (e.g. NH₃R⁺, NH₂R₂⁺, NHR₃⁺, NR₄⁺, where R is an alkyl group).

If the compound has both a cationic functional group, or a functional group that can become cationic, and an anionic functional group, or a functional group that can become anionic, then the compound may be present as a zwitterion.

The term “hydrogen” or “hydrogen atom” as used herein refers to a —H moiety.

The term “halo”, “halogen” or “halogen atom” as used herein refers to a —F,-C1, —Br or —I moiety.

The term “hydroxy” as used herein refers to an —OH moiety.

The term “alkyl” as used herein refers to a monovalent moiety obtained by removing a hydrogen atom from a carbon atom of a hydrocarbon compound having from 1 to 12 carbon atoms (unless otherwise specified), which may be aliphatic or alicyclic, which may be saturated or unsaturated (e.g. partially unsaturated or fully unsaturated), and which may be linear or branched. Thus, the term “alkyl” includes the sub-classes alkenyl, alkynyl, cycloalkyl, cycloalkenyl and cylcoalkynyl below.

In the context of alkyl groups, the prefix C1-12 denotes the number of carbon atoms, or range of number of carbon atoms present in that group. Thus, the term “C₁₋₁₂ alkyl” refers to an alkyl group having from 1 to 12 carbon atoms. The first prefix may vary according to the nature of the alkyl group. Thus, if the alkyl group is an alkenyl or alkynyl group, then the first prefix must be at least 2 (e.g. C₂₋₁₂). For cyclic (e.g. cycloalkyl, cycloalkenyl, cylcoalkynyl) or branched alkyl groups, the first prefix must be at least 3 (e.g. C₃₋₁₂).

Examples of saturated alkyl groups include methyl (C₁), ethyl (C₂), propyl (C₃), butyl (C₄), pentyl (C₅), hexyl (C₆), heptyl (C₇), octyl (C₈), nonyl (C₉) and decyl (C₁₀). Examples of saturated linear alkyl groups include, but are not limited to, methyl (CO, ethyl (C₂), n-propyl (C₃), n-butyl (C₄), n-pentyl (amyl) (C₅), n-hexyl (C₆), and n-heptyl (C₇). Examples of saturated branched alkyl groups include iso-propyl (C₃), iso-butyl (C₄), sec-butyl (C₄), tert-butyl (C₄), iso-pentyl (Cₛ), and neo-pentyl (C₅).

The term “alkenyl” refers to an alkyl group having one or more carbon-carbon double bonds. Examples of unsaturated alkenyl groups include ethenyl (vinyl, —CH═CH₂), 1-propenyl (—CH═CH—CH₃) and 2-propenyl (allyl,-CH-CH═CH₂).

The term “alkynyl” refers to an alkyl group having one or more carbon-carbon triple bonds. Examples of unsaturated alkynyl groups include, but are not limited to, ethynyl (ethinyl,-CECH) and 2-propynyl (propargyl, —CH₂—C≡CH).

The term “cycloalkyl” refers an alkyl group which is also a cyclyl group; that is, a monovalent moiety obtained by removing a hydrogen atom from an alicyclic ring atom of a carbocyclic compound (i.e. a compound where all of the ring atoms are carbon atoms). The ring may be saturated or unsaturated (e.g. partially unsaturated or fully unsaturated), which moiety has from 3 to 12 carbon atoms (unless otherwise specified). Thus, the term “cycloalkyl” includes the sub-classes cycloalkenyl and cycloalkynyl. In an embodiment, each ring has from 3 to 7 ring carbon atoms. Examples of cycloalkyl groups include those derived from (i) saturated monocyclic hydrocarbon compounds: cyclopropane (C₃), cyclobutane (C₄), cyclopentane (C₅), cyclohexane (C₆), cycloheptane (C₇) and methylcyclopropane (C₄); (ii) unsaturated monocyclic hydrocarbon compounds: cyclopropene (C₃), cyclobutene (C₄), cyclopentene (C₅), cyclohexene (C₆), methylcyclopropene (C₄) and dimethylcyclopropene (C₅); (iii) saturated polycyclic hydrocarbon compounds: thujane (C₁₀), carane (C₁₀), pinane (C₁₀), bornane (C₁₀), norcarane (C₇), norpinane (C₇), norbornane (C₇), adamantane (C₁₀), decalin (C₁₀); (iv) unsaturated polycyclic hydrocarbon compounds: camphene (C₁₀), limonene (C₁₀), pinene (C₁₀); and (v) polycyclic hydrocarbon compounds having an aromatic ring: indene (C₉), indane (C₉) and tetraline (C₁₀).

In an embodiment, a reference to an alkyl group described herein is a C₁₋₁₂ alkyl group, such as a C₁₋₈ alkyl group, for example a C₁₋₆ alkyl group, or a C₁₋₄ alkyl group. The alkyl groups in the invention can be saturated alkyl groups or saturated cycloalkyl groups, for example saturated, unbranched alkyl groups.

The phrase “optionally substituted” as used herein refers to a parent group which may be unsubstituted or which may be substituted with one or more, for example one or two, substituents. The substituents on an “optionally substituted” group may for example be selected from alkyl, cycloalkyl, alkenyl, alkynyl, aryl, heteroaryl and heterocyclyl groups; carboxylic acids and carboxylate ions; carboxylate esters; carbamates; alkoxyl groups; ketone and aldehyde groups; amine and amide groups; —OH; —CN; —NO₂; and halogens.

The term “substituents” is used herein in the conventional sense and refers to a chemical moiety, which is covalently attached to, or if appropriate, fused to, a parent group.

In some embodiments, substituents can themselves be substituted. For example, a C₁₋₁₂alkyl group may be substituted with, for example, hydroxy (referred to as a hydroxy-C₁₋₁₂alkyl group) or a halogen atom (referred to as a halo-C₁₋₁₂ alkyl group), and a C₁-12alkoxy group may be substituted with, for example, a halogen atom (referred to as a halo-C₁-12alkoxy group).

The term “aryl” as used herein refers to a monovalent moiety obtained by removing a hydrogen atom from an aromatic ring atom of an aromatic compound, which moiety has from 6 to 10 ring carbon atoms (unless otherwise specified). In an embodiment, the aryl group is a phenyl group.

The term “heteroaryl” as used herein refers to a monovalent moiety obtained by removing a hydrogen atom from a heteroaromatic compound, which moiety may for example be a monocyclic or bicyclic group. The heteroaryl moiety may contain from 1 to 12 carbon atoms (unless otherwise specified) and one or more N, O or S atoms. The heteroaryl moiety may be a 5 or 6-membered ring containing one or more N atoms.

The term “heterocyclyl” as used herein refers to a monovalent moiety obtained by removing a hydrogen atom from a ring atom of a heterocyclic compound, which moiety may for example be a monocyclic or bicyclic group. The heterocyclyl group may contain from 1 to 12 carbon atoms (unless otherwise specified) and one or more N, O or S atoms.

The term “alkoxy” used herein refers to an alkyl-oxy group, where the alkyl group is as defined above and has from 1 to 12 carbon atoms (unless otherwise specified). In an embodiment, the alkyl moiety in an alkoxy group is a saturated alkyl group or a saturated cycloalkyl group. In an embodiment, the alkyl moiety is a saturated, unbranched alkyl group. Examples of C₁₋₁₂ alkoxy groups include —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

The term “phosphinic acid moiety” used herein refers to a functional group containing a P(O)OH group.

The term “phosphinate ester moiety” used herein refers to an ester of a phosphinic acid moiety, i.e. a phosphinic acid moiety wherein the hydrogen of the acid (P(0)OH) group has been replaced by an organic substituent, for example an alkyl, cycloalkyl, aryl, heteroaryl, alkenyl or alkynyl group (e.g. an alkyl group).

Where present, the functional groups C(O)O and C(O)NR can be found in either orientation. In other words, C(O)O represents —C(O)O— and —OC(O)—; and C(O)NR represents —C(O)NR— and —NRC(0)—.

Certain compounds may exist in one or more particular geometric, enantiomeric, diasteriomeric, tautomeric, or conformational forms. Unless otherwise specified, a reference to a particular compound includes all such isomeric forms, including (wholly or partially) racemic and other mixtures thereof. Methods for the preparation and separation of such isomeric forms are either known in the art.

The term “subject” used herein includes humans, non-human animals (e.g. dogs, cats, rabbits, cattle, horses, sheep, goats, swine, deer and the like) and non-mammals (e.g. birds and the like).

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

The present invention provides a compound for use in the treatment of nephrotic syndrome in a subject in need thereof. Preferably, the present invention provides a compound for use in the treatment of nephrotic syndrome in a subject in need thereof, wherein the compound is an inhibitor of binding between a target protein and keratin 8.

Without wishing to be bound by theory, the inventors have identified the mechanisms in podocyte cells whereby certain proteins involved in nephrotic syndrome are prevented from reaching their target site. These mechanisms involve binding by keratin 8, which leads to retention of the protein-keratin 8 complex in the podocyte endoplasmic reticulum and ultimately proteasomal degradation chaperoned by keratin 8. The inventors have further identified that the binding of these proteins by keratin 8 can be disrupted by inhibitors, leading to restoration of these proteins at their podocyte target sites and ultimately the restoration of kidney function. These compounds represent a first in class opportunity for the treatment of nephrotic syndrome, and in particular offer a real opportunity for the treatment of steroid resistant nephrotic syndrome.

Preferably, the target protein is podocin. Preferably, the target protein is mutated podocin. The mutated podocin may have a truncating or a missense mutation. The truncating mutation may be nonsense or frameshift. Preferably the mutation is one which causes the podocin to bind keratin 8 (e.g. by causing a hydrophobic patch within the podocin which binds keratin 8). An example of such a mutation is the R138Q mutation. The target protein is, therefore, preferably podocin with the R138Q mutation (also described herein as “R138Q podocin”). The podocin may preferably be homozygous p.R138Q podocin.

The compounds described herein may be used in the treatment of nephrotic syndrome (NS). The nephrotic syndrome is preferably steroid-resistant nephrotic syndrome (SRNS). The nephrotic syndrome is preferably genetic nephrotic syndrome (e.g. SRNS with pathologically prevalent focal segmental glomerulosclerosis (FSGS)). The genetic nephrotic syndrome may preferably be autosomal recessive SRNS.

Preferably, the nephrotic syndrome (NS) is NS associated with (e.g. caused by) a podocin mutation. In particular, the NS is NS associated with (e.g. caused by) a mutation that causes binding with keratin 8. More preferably, the NS is NS associated with (e.g. caused by) an R138Q podocin mutation.

Preferably, the compound comprises at least one moiety selected from phosphinic acid moieties, phosphinate ester moieties and pharmaceutically acceptable salts thereof. It has surprisingly been found that compounds comprising at least one phosphinic acid moiety and/or phosphinate ester moiety are effective in the treatment of nephrotic syndrome. Without wishing to be bound by theory, it is believed that compounds comprising such moieties disrupt the binding of keratin 8 to proteins such as podocin leading to restoration of these proteins at their target sites.

Thus, the present invention provides a compound for use in the treatment of nephrotic syndrome in a subject in need thereof, wherein the compound comprises at least one moiety selected from phosphinic acid moieties, phosphinate ester moieties and pharmaceutically acceptable salts thereof.

Pharmaceutically acceptable salts of the compound for use according to the present invention may be of any suitable type. Preferred pharmaceutically acceptable salts are alkali metal salts, such as sodium and potassium salts.

A preferred compound for use in the treatment of nephrotic syndrome is a compound of formula I, or a pharmaceutically acceptable salt thereof:

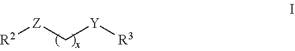

wherein x is 0 or an integer from 1 to 6; Y is selected from the group consisting of: direct bond, C(0), C(O)O, 0, C(R′)(OH), C(O)NR^{I}, S(0), S(0)(0) and P(0)(OR′);

Z is selected from the group consisting of: C(0), C(O)O, 0, C(R′)(OH), C(O)NR^{I}, S(0), S(0)(0) and P(0)(OR′);

wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;

R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl; and

R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

A particularly preferred compound for use in the treatment of nephrotic syndrome is a compound of formula II, or a pharmaceutically acceptable salt thereof:

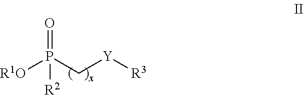

wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;

R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;
x is 0 or an integer from 1 to 6;

Y is selected from the group consisting of: direct bond, C(0), S(0), C(O)O, C(O)NH, and P(O)(OH); and

R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

In the compound of formula I and/or the compound of formula II, R¹ is selected from the group consisting of: hydrogen, optionally substituted alkyl (e.g. preferably unsubstituted C₁₋₁₂ alkyl or preferably unsubstituted C₁-6 alkyl), optionally substituted cycloalkyl (e.g. preferably unsubstituted C₃₋₁₂ cycloalkyl or preferably unsubstituted C₃₋₆ cycloalkyl), optionally substituted alkenyl (e.g. preferably unsubstituted C₁₋₁₂ alkenyl or preferably unsubstituted C₁-6 alkenyl), optionally substituted alkynyl (e.g. preferably unsubstituted C₁-12 alkynyl or preferably unsubstituted C₁-6 alkynyl), optionally substituted aryl (e.g. preferably unsubstituted C₆₋₁₂ aryl or preferably unsubstituted C₆ aryl), optionally substituted heteroaryl (e.g. preferably unsubstituted C₅₋₁₂ heteroaryl or preferably unsubstituted C₅₋₆ heteroaryl), and optionally substituted heterocyclyl (e.g. preferably unsubstituted C₃₋₁₂ heterocyclyl or preferably unsubstituted C₃₋₆ heterocyclyl). Preferably R¹ is selected from the group consisting of: hydrogen, optionally substituted alkyl and optionally substituted cycloalkyl, more preferably, hydrogen and optionally substituted alkyl. Still more preferably R¹ is selected from the group consisting of: hydrogen and optionally substituted alkyl (e.g. unsubstituted C₁₋₁₂ alkyl). Preferred alkyl groups include methyl, ethyl, propyl (e.g. n-propyl, i-propyl), butyl (e.g. n-butyl, i-butyl, s-butyl, t-butyl), pentyl, hexyl and heptyl, more preferably methyl, ethyl, propyl and butyl.

In the compound of formula I and/or the compound of formula II, R² is selected from the group consisting of: hydrogen, optionally substituted alkyl (e.g. preferably unsubstituted C₁₋₁₂ alkyl or preferably unsubstituted C₁-6 alkyl), optionally substituted cycloalkyl (e.g. preferably unsubstituted C₃₋₁₂ cycloalkyl or preferably unsubstituted C₃₋₆ cycloalkyl), optionally substituted alkenyl (e.g. preferably unsubstituted C₁₋₁₂ alkenyl or preferably unsubstituted C₁-6 alkenyl), optionally substituted alkynyl (e.g. preferably unsubstituted C₁₋₁₂ alkynyl or preferably unsubstituted C₁-6 alkynyl), optionally substituted aryl (e.g. preferably unsubstituted C₆₋₁₂ aryl or preferably unsubstituted C₆ aryl), optionally substituted heteroaryl (e.g. preferably unsubstituted C₅₋₁₂ heteroaryl or preferably unsubstituted C₅₋₆ heteroaryl), and optionally substituted heterocyclyl (e.g. preferably unsubstituted C₃₋₁₂ heterocyclyl or preferably unsubstituted C₃₋₆ heterocyclyl). Preferably R²is selected from the group consisting of: optionally substituted aryl (e.g. C₆₋₁₂ aryl) and optionally substituted heteroaryl (e.g. C₅₋₁₂ heteroaryl). Preferred aryl groups include optionally substituted phenyl and optionally substituted naphthyl groups, more preferably optionally substituted phenyl. Preferred heteroaryl groups include pyrryl, furyl, thiophenyl, pyridinyl, indolyl and imidazolyl. When substituted, preferred substituents include halo (e.g. fluoro, chloro, bromo, iodo), hydroxy, C₁₋₁₂ alkyl, C₁₋₁₂ alkoxy (e.g. methoxy, ethoxy, propoxy, butoxy), COOH, carboxylate ester, nitrile, nitro and amine, more preferably halo (e.g. fluoro, chloro, bromo, iodo), hydroxy, C₁₋₁₂ alkyl, C₁₋₁₂ alkoxy (e.g. methoxy, ethoxy, propoxy, butoxy) and COOH, still more preferably halo. R² is especially preferably unsubstituted or substituted phenyl (e.g. halophenyl such as fluorophenyl, chlorophenyl, bromophenyl, iodophenyl). R² may preferably be unsubstituted phenyl.

In the compound of formula I and/or the compound of formula II, x is 0 or an integer from 1 to 6 (e.g. 1, 2, 3, 4, 5 or 6). Preferably, x is 0 or an integer from 1 to 3. More, preferably,
x is 1, 2 or 3 (e.g. 1 or 2).

In the compound of formula I, Y is selected from the group consisting of: direct bond, C(0),

C(O)O, 0, C(R′)(OH), C(O)NR^{I}, S(0), S(0)(0) and P(0)(OR′). In the compound of formula II, Y is selected from the group consisting of: direct bond, C(0), S(0), and P(O)(OH). Preferably, Y is selected from the group consisting of: direct bond, C(0), and P(O)(OH). Y may preferably be P(O)(OH).

In the compound of formula I and/or the compound of formula II, R³ is selected from the group consisting of: hydrogen, OH, optionally substituted alkyl (e.g. preferably unsubstituted C₁₋₁₂ alkyl or preferably unsubstituted C₁-6 alkyl), optionally substituted cycloalkyl (e.g. preferably unsubstituted C₃₋₁₂ cycloalkyl or preferably unsubstituted C₃₋₆ cycloalkyl), optionally substituted alkenyl (e.g. preferably unsubstituted C₁._1.2 alkenyl or preferably unsubstituted C₁-6 alkenyl), optionally substituted alkynyl (e.g. preferably unsubstituted C₁₋₁₂ alkynyl or preferably unsubstituted C₁-6 alkynyl), optionally substituted aryl (e.g. preferably unsubstituted C₆₋₁₂ aryl or preferably unsubstituted C₆ aryl), optionally substituted heteroaryl (e.g. preferably unsubstituted C₅₋₁₂ heteroaryl or preferably unsubstituted C₅₋₆ heteroaryl), optionally substituted heterocyclyl (e.g. preferably unsubstituted C₃₋₁₂ heterocyclyl or preferably unsubstituted C₃₋₆ heterocyclyl)., optionally substituted alkoxy (e.g. preferably unsubstituted C₁-6 alkoxy), COOH, optionally substituted carboxylate ester (e.g. preferably unsubstituted C₁-6 carboxylate ester), optionally substituted amide (e.g. preferably unsubstituted C₁-6 carboxylate amide), optionally substituted amine, optionally substituted ether (e.g. preferably unsubstituted C₁-6 ether), phosphinic acid (e.g. P(O)(OH)R) and phosphinate ester (e.g. P(0)(OR′)R). Preferably, R³ is selected from the group consisting of: OH, optionally substituted alkyl (e.g. preferably unsubstituted C₁₋₁₂ alkyl or preferably unsubstituted C₁-6 alkyl), optionally substituted cycloalkyl (e.g. preferably unsubstituted C₃₋₁₂ cycloalkyl or preferably unsubstituted C₃₋₆ cycloalkyl), optionally substituted aryl (e.g. preferably unsubstituted C₆₋₁₂ aryl or preferably unsubstituted C₆ aryl), optionally substituted heteroaryl (e.g. preferably unsubstituted C₅₋₁₂ heteroaryl or preferably unsubstituted C₅₋₆ heteroaryl), and optionally substituted alkoxy (e.g. preferably unsubstituted C₁-6 alkoxy). More preferably, R³ is selected from the group consisting of: OH, optionally substituted (e.g. unsubstituted) C₁-6 alkyl and optionally substituted C₆₋₁₂ aryl (e.g. substituted or unsubstituted phenyl). Preferred alkyl groups include methyl, ethyl, propyl (e.g. n-propyl, i-propyl), butyl (e.g. n-butyl, i-butyl, s-butyl, t-butyl), pentyl, hexyl and heptyl, more preferably methyl, ethyl, propyl and butyl. When substituted, preferred substituents include halo (e.g. fluoro, chloro, bromo, iodo), hydroxy,

C₁₋₁₂ alkyl, C₁₋₁₂ alkoxy (e.g. methoxy, ethoxy, propoxy, butoxy), COOH, carboxylate ester, nitrile, nitro and amine. R³ is especially preferably hydroxy, unsubstituted C₁-6 alkyl or optionally substituted phenyl (e.g. unsubstituted phenyl, halophenyl or hydroxyphenyl, preferably unsubstituted phenyl or hydroxyphenyl).

In a particularly preferred compound of formula II for use according to the invention:

R¹ is selected from hydrogen, optionally substituted C₁₋₁₂ alkyl and optionally substituted C₃-12 cycloalkyl;

R² is selected from optionally substituted C₆₋₁₂ aryl and optionally substituted C₅₋₁₂ heteroaryl;

Y is selected from the group consisting of: direct bond, C(0), S(0), C(O)O, C(O)NH, and

P(O)(OH); and

R³ is selected from the group consisting of: hydrogen, optionally substituted C₁₋₁₂ alkyl, optionally substituted C₃₋₁₂ cycloalkyl, optionally substituted C₆₋₁₂ aryl, optionally substituted C₅-12 heteroaryl, COOH, optionally substituted carboxylate ester, phosphinic acid and phosphinate ester;

wherein the optional substituents are selected from halo, hydroxy, C₁₋₁₂ alkyl, C₁₋₁₂ alkoxy, COOH, carboxylate ester, nitrile, nitro and amine.

An especially preferred compound of formula II for use in the treatment of nephrotic syndrome is one wherein:

R¹ is selected from hydrogen and C₁-6 alkyl (e.g. methyl, ethyl, propyl, butyl, pentyl; hexyl);

R² is selected from optionally substituted C₆₋₁₂ aryl and optionally substituted C₅₋₁₂ heteroaryl;

x is an integer from 1 to 3;

Y is selected from the group consisting of: P(O)(OH); and

R³ is selected from the group consisting of: hydrogen, C₁-6 alkyl, optionally substituted phenyl, COOH, and phosphinic acid;

wherein the optional substituents are selected from halo, hydroxy, C₁₋₁₂ alkyl, C₁₋₁₂ alkoxy, COOH, carboxylate ester, nitrile, nitro and amine (e.g. halo, hydroxy and alkoxy).

Especially preferred compounds for use in the treatment of nephrotic syndrome in a subject in need thereof are selected from compounds of formulae IIa-IIh:

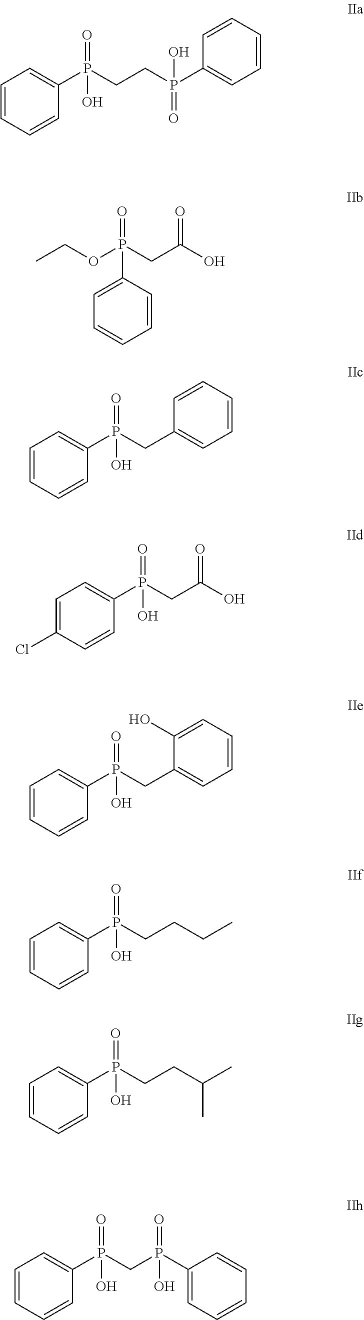

A particularly preferred compound is the compound of formula IIa.

In one embodiment, the compound is other than a bisphosphonate drug. For example, the compound is other than risedronic acid, etidronic acid, alendronic acid, minodronic acid, zoledronic acid, pamidronic acid, tildronic acid, monidronic acid, neridronic acid, olpadronic acid, clodronic acid and ibandronic acid. In one embodiment, the compound does not have one or two or more terminal phosphonate (P(O)(OH)₂) functional groups.

The present invention provides a compound as hereinbefore described for use in the treatment of nephrotic syndrome in a subject in need thereof. Preferably the subject is a mammal, more preferably a human.

The present invention also provides a kit comprising a compound which is an inhibitor of binding between a target protein and keratin 8 together with instructions for treating nephrotic syndrome. Preferred features of the kit, including preferred features of the compound, target protein, nephrotic syndrome and subject, are as defined above with respect to the compound for use in the treatment of nephrotic syndrome.

The present invention also provides a method of treating nephrotic syndrome in a subject in need thereof comprising administering to said subject an effective amount of a compound which is an inhibitor of binding between a target protein and keratin 8. Preferred features of the method, including preferred features of the compound, target protein, nephrotic syndrome and subject, are as defined above with respect to the compound for use in the treatment of nephrotic syndrome.

The therapeutically effective amount of the compound administered to the patient is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect).

The specific therapeutically effective dose level for any particular patient will depend upon a variety of factors including the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The compounds may be administered in any effective manner. Suitable examples of the administration form include without limitation oral, topical, parenteral, sublingual, rectal, vaginal, ocular, and intranasal. Parenteral administration includes subcutaneous injections, intravenous, intramuscular, intrasternal injection or infusion techniques.

The present invention also provides the use of a compound which is an inhibitor of binding between a target protein and keratin 8 in the manufacture of a medicament for the treatment of nephrotic syndrome in a subject in need thereof. Preferred features of the method, including preferred features of the compound, target protein, nephrotic syndrome and subject, are as defined above with respect to the compound for use in the treatment of nephrotic syndrome.

### Examples

The present work describes previously unknown machinery that plays role in the R138Q podocin intracellular retention and firmly suggests that keratin 8-R138Q podocin association facilitates the ER retention of the mutant protein. Keratins have been previously reported to be up-regulated in different disease models, such as animal models of pancreas and liver injury, while keratin 8 and 18 have been recently described to be novel markers of renal epithelial cell injury (S. Djudjaj et al., Kidney Int., vol. 89, pp. 792-808, 2016.). The current study reports an increased expression of keratin 8 in R138Q mutant human podocytes for the first time. There is also an increased interaction of keratin 8 and podocin in close vicinity of the ER in R138Q mutant cells, which is reversed by treating cells with compound Ia, resulting in the rescue of the mutant protein localization.

A full rescue of adhesion function of mutant cells was achieved upon treatment with compound Ia. Furthermore, computer modelling of protein folding revealed that R138Q mutation creates a large hydrophobic region, which becomes exposed for interaction with other proteins or small molecules, such as compound Ia. Therefore, it is tempting to hypothesize that K8-R138Q interaction occurs as a result of the appearance of this hydrophobic pocket, and further structural modelling shows that inhibitor compounds are able to bind to this pocket blocking the interaction with K8 and thus allowing for the correction of the R138Q podocin processing defect with small molecule intervention.

Here, we demonstrate that keratin 8 shRNA results in the correct localization of the R138Q mutant protein and the recovery of its function. The fact that silencing of keratin 8 recovers the function of the mutant protein further suggests that keratin 8 network can act as a potential therapeutic target.

In summary, our study provides direct evidence that keratin 8 is involved in the podocyte dysfunction associated with the most common R138Q podocin mutation in NS. This work further demonstrates that pharmacological targeting of keratin 8 with inhibitor compounds, such as compound Ia, in vivo leads to prevention and correction of proteinuria in NPHS2^{flox/R140Q} mice.

### Example 1—Intracellular Localization of R138Q Podocin

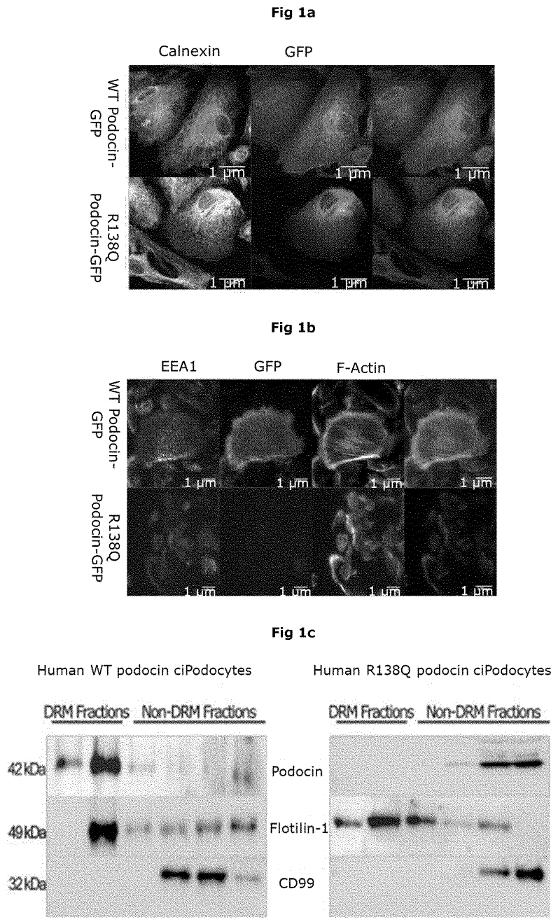

In this work, the consequence of the R138Q mutation of podocin was studied by expressing the GFP-tagged wild-type and R138Q mutant podocins in human podocytes. As shown in FIG. 1a, wild-type podocin displayed a localization consisting of a plasma membrane staining and an intracellular vesicular labelling. On the other hand, the R138Q podocin mutant was strongly retained in the ER as seen by complete colocalization with calnexin, an ER marker. Furthermore, TIRF microscopy was used to access podocin's localization at or near the plasma membrane in human podocytes. The fluorescence signal obtained by TIRF imaging represents podocin within 100 nm of the coverslip, i.e., in or in close proximity to the plasma membrane. The mutant podocin displayed no plasma membrane labelling, while wild-type podocin localized with F-Actin at the plasma membrane (FIG. 1b). Next, flotation gradient centrifugation was used to determine whether the disease-causing mutation of podocin interfered with its ability to associate with lipid rafts. In flotation gradients of Triton X-100 extracts, detergent-insoluble raft-associated proteins represent the first 4 fractions, while detergent-soluble protein/protein complexes are identified as fractions 5-8, 9-12. The quality and purity of the fractions were examined using Flotillin-1, as a lipid raft marker. As expected, the R138Q podocin was not able to associate with lipid rafts due to its ER retention, causing lipid raft composition to be altered, while wild-type podocin reached the plasma membrane and associated with detergent-resistant membranes (FIG. 1c).

### Example 2—Keratin 8/18 and Podocin

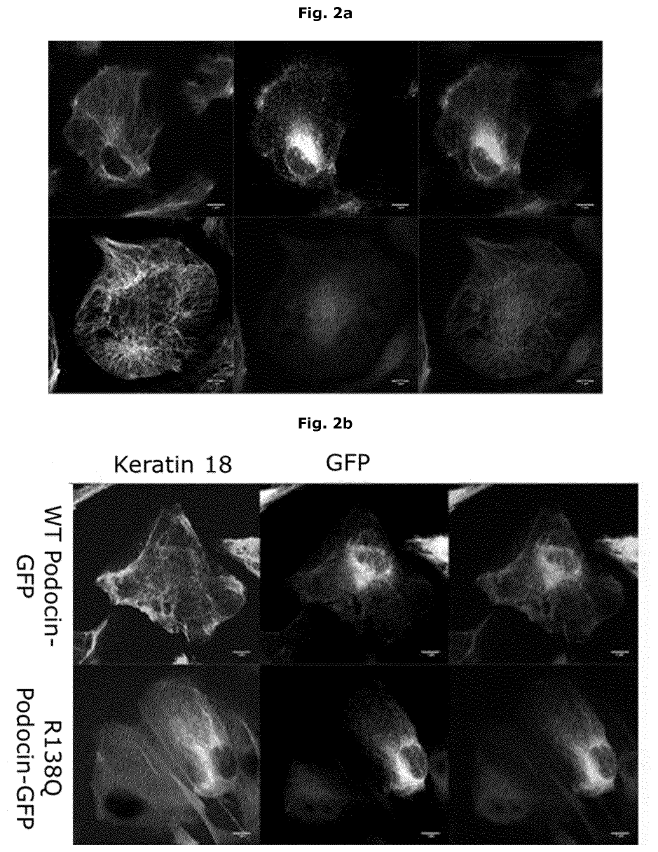

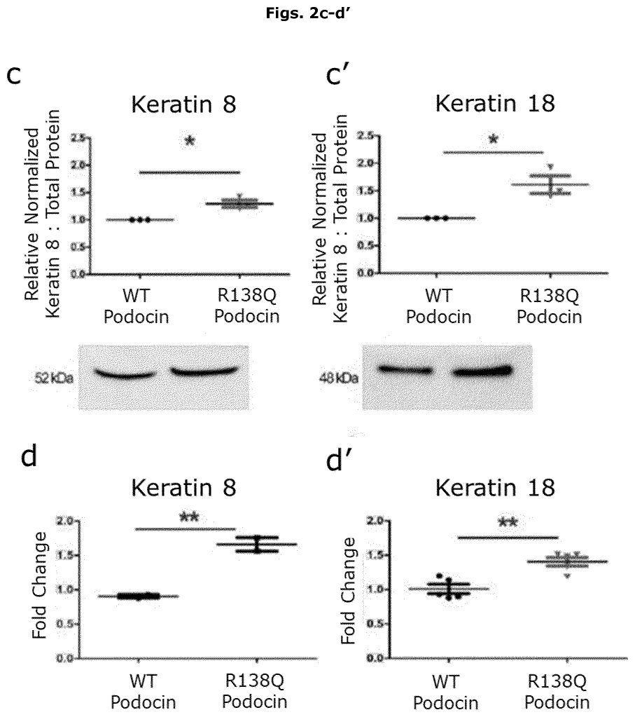

In the next set of experiments, the intracellular distribution of keratin 8/18 was analysed in human podocytes expressing either the GFP-tagged wild-type or R138Q mutant podocins using confocal microscopy. Localization of keratin 8 and WT podocin was shown by immunofluorescence at the cytosol level, while localization of keratin 8 was seen in the perinuclear area in close vicinity of R138Q podocin (FIG. 2a). Here, the keratin 8 expression was elevated, and the keratin 8 network reorganized itself to form the meshwork like structure, further trapping the mutant protein in the ER. The same was observed for Keratin 18 (FIG. 2b). Next, an immunoblot and qPCR analyses were performed to verify the expression of K8 and K18 on both the protein and RNA levels in human podocytes. The results confirmed the observation of the immunofluorescence analysis, both K8/K18 protein and RNA expression levels were significantly higher in human podocytes derived from the patient bearing R138Q mutation (FIG. 2c, d).

### Example 3—Keratin 8-R138Q Podocin Interaction

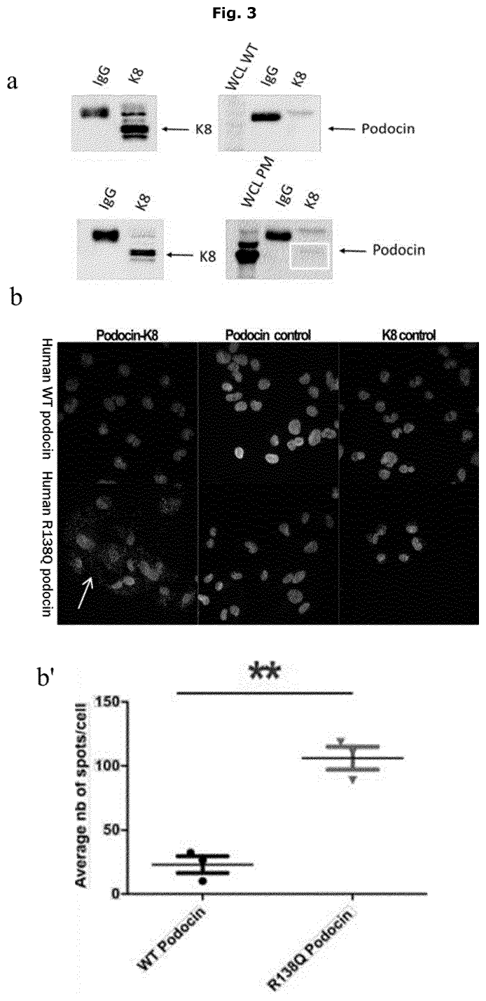

Co-immunoprecipitation (Co-IP) assay of endogenous proteins was then conducted to see whether keratin 8 and podocin interact directly in human podocytes. Co-immunoprecipitation experiments demonstrated that endogenous keratin 8 preferably interacts with R138Q podocin over its wild-type counterpart (FIG. 3a). To further search for K8-Podocin interaction in vitro, the Proximity Ligation Assay (PLA) was performed using the Duolink™ kit (Eurogentec, Angers, France) according to manufacturer's instructions in human podocytes derived from either a healthy patient or a patient with R138Q mutation (J. Colas et al., Hum. Mol. Genet., vol. 21, no. 3, pp. 623-34, Feb. 2012). The PLA is a highly sensitive and specific approach that can directly detect proteins and protein interactions within 40 nm proximity in unmodified cells. A<40 nm proximity between K8 and R138Q podocin was identified in human podocytes (FIG. 3b). This strongly suggests that detectable physical interaction between K8 and podocin only prevails for the R138Q mutation.

### Example 4—Silencing of Keratin 8 Restores R138Q Podocin's Localization and Function in Patient's Cells

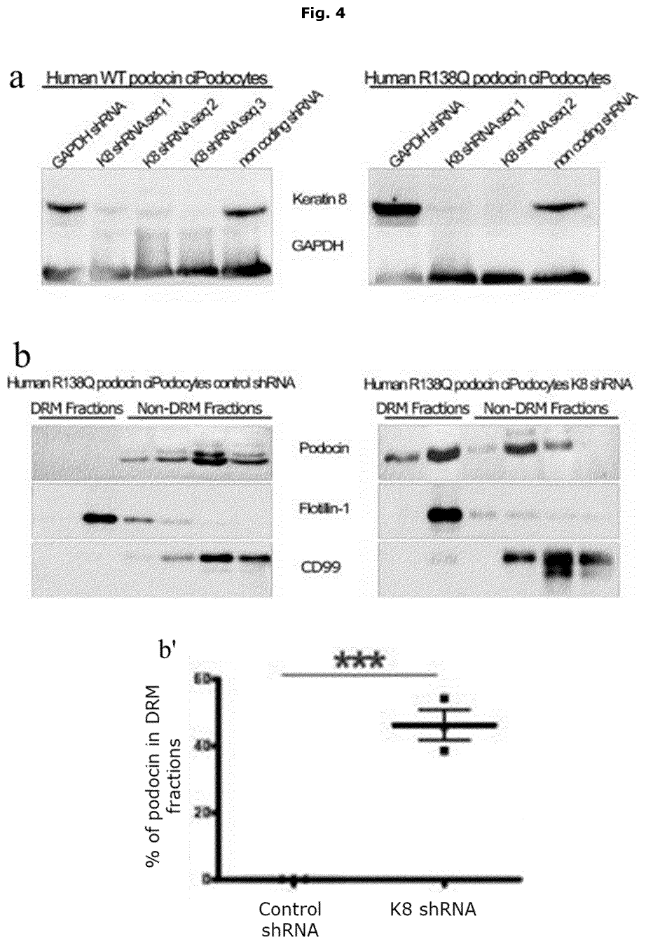

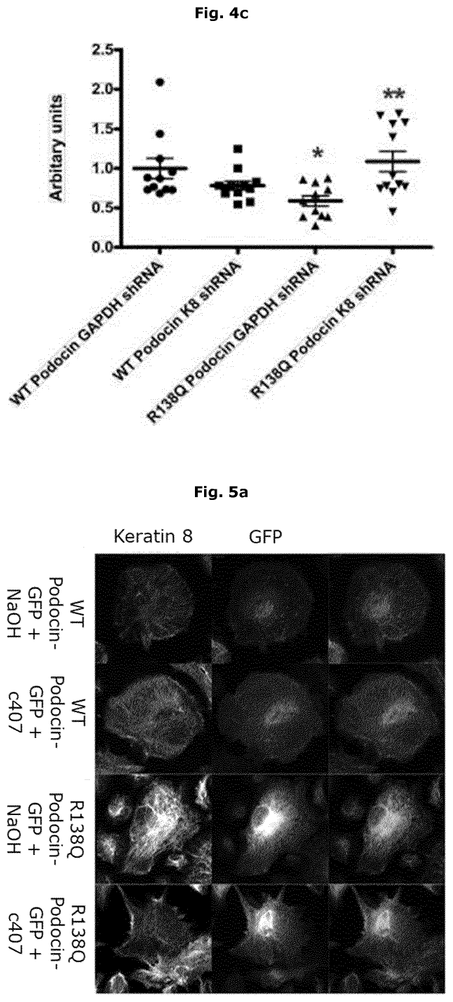

It was observed that inhibiting the keratin 8-R138Q podocin interaction by keratin 8 silencing rescued the lipid raft association of the mutant protein and restore its functional defect. All K8-shRNA sequences considerably decreased K8 expression in both human podocyte cell lines (FIG. 4a). The effect of keratin 8-shRNA on lipid raft association of mutant podocin was studied in patient's cells. Knockdown of keratin 8 in mutant podocytes restored the association of the R138Q podocin with lipid rafts, while non-coding shRNA had no effect (FIG. 4b). Finally, to evaluate the functional impact of keratin 8 silencing, human K8 shRNA podocyte cells were further analysed for their adhesive capacity in an in vitro adhesive assay. There was a significant decrease in adhesion observed in GAPDH shRNA mutant podocyte cell line, when compared to the wild-type control. This adhesive defect of the mutant podocyte cell line was rescued by knock-out of K8 (FIG. 4c).

### Example 5— Compound Ia disrupts Keratin 8-R138Q podocin interaction

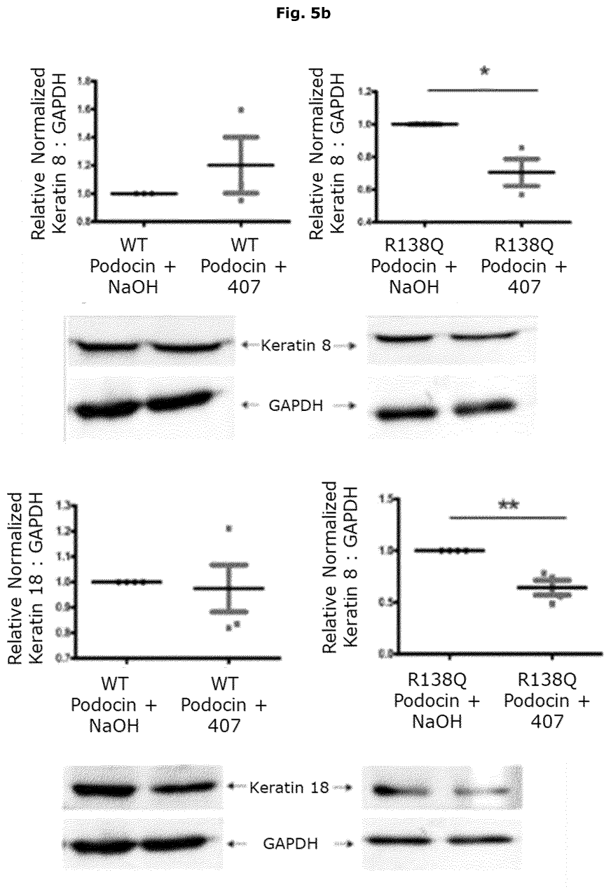

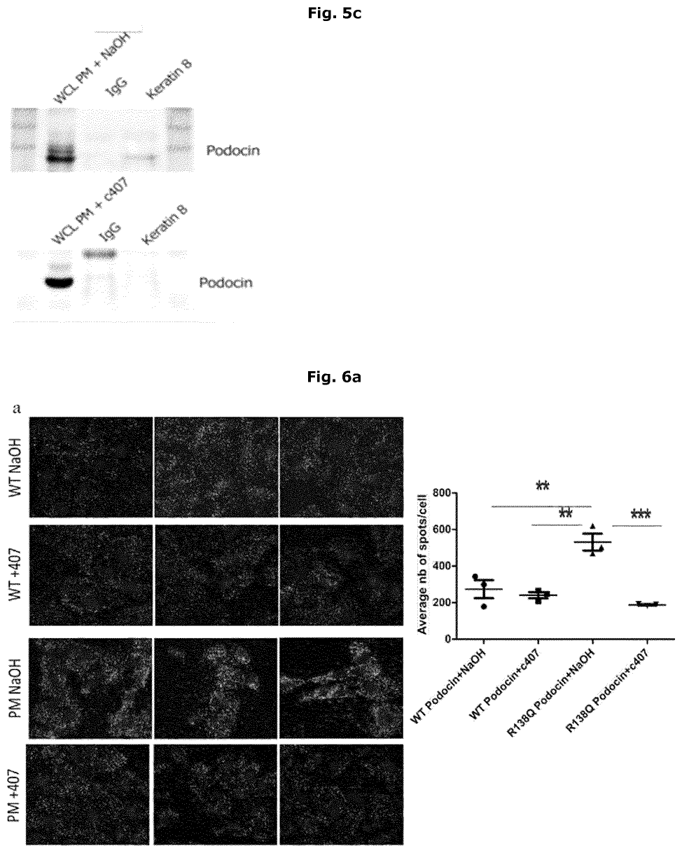

Immunofluorescence studies were performed to see whether treatment with compound Ia for 24 hours rescued mutant podocin localization back to the plasma membrane. FIG. 5a shows immunofluorescence images of Podocin (green) and Keratin 8 (red) in human podocytes stably expressing either GFP-tagged WT podocin or R138Q podocin mutant in control conditions and after treatment with compound Ia. Treatment with compound Ia rescued the localization of mutant podocin back to the plasma membrane. Furthermore, compound Ia treatment decreased both keratin 8 and 18 protein expression levels detected by Western Blotting (FIG. 5b). Next, to test the theory human podocytes derived from the patient with the R138Q mutation were treated with compound Ia to see whether it caused the targeted disruption of the protein-protein interaction in vitro. As expected, compound Ia interrupted the K8-R138Q podocin interaction, while treatment with NaOH had no effect, where the K8-R138Q podocin interaction was observed using Co-IP (FIG. 5c).

### Example 6—Inhibition of K8-R138Q Podocin Interaction by Compound Ia Restores Function of the Mutant Protein

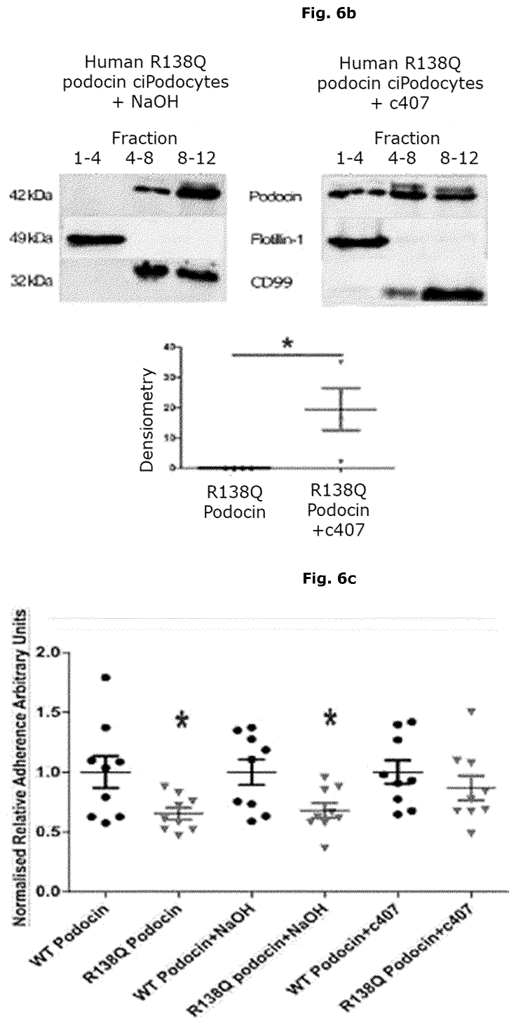

To test whether compound Ia interrupts the protein-protein interaction, a series of proximity ligation experiments were performed on human podocytes derived either from a healthy patient or patient with the R138Q mutation. FIG. 6a demonstrates that there is a significant decrease in K8-R138Q podocin interaction, indicated by green fluorescent spots, after treating cells with compound Ia. Furthermore, that disruption of interaction results in functional correction of the mutant protein as seen by two sets of experiments: (a) lipid raft isolation in human mutant podocytes and (b) an adhesion assay. Subsequent treatment with compound Ia allowed the association of mutant podocin with specialized lipid raft microdomains of the plasma membrane, while also restored the lipid raft composition (FIG. 6b). Finally, the adhesive defect of the mutant cells was corrected with compound Ia, while treatment with NaOH had no effect (FIG. 6c).

### Example 7— Compound Ia Treatment Prevents the Development of NS in the Transgenic Mouse Model of the R140Q Mutation

To understand whether compound Ia could prevent the development of NS in NPHS2^{flox/R140Q} mice, doxycycline was administrated for 3 weeks to induce proteinuria, at the same time as one group was treated with compound Ia and another group was treated with vehicle (0.9% NaCl) for 4 weeks via osmotic mini pumps. A third group with vehicle pumps served as no disease controls and was administrated drinking water for the duration of the study.

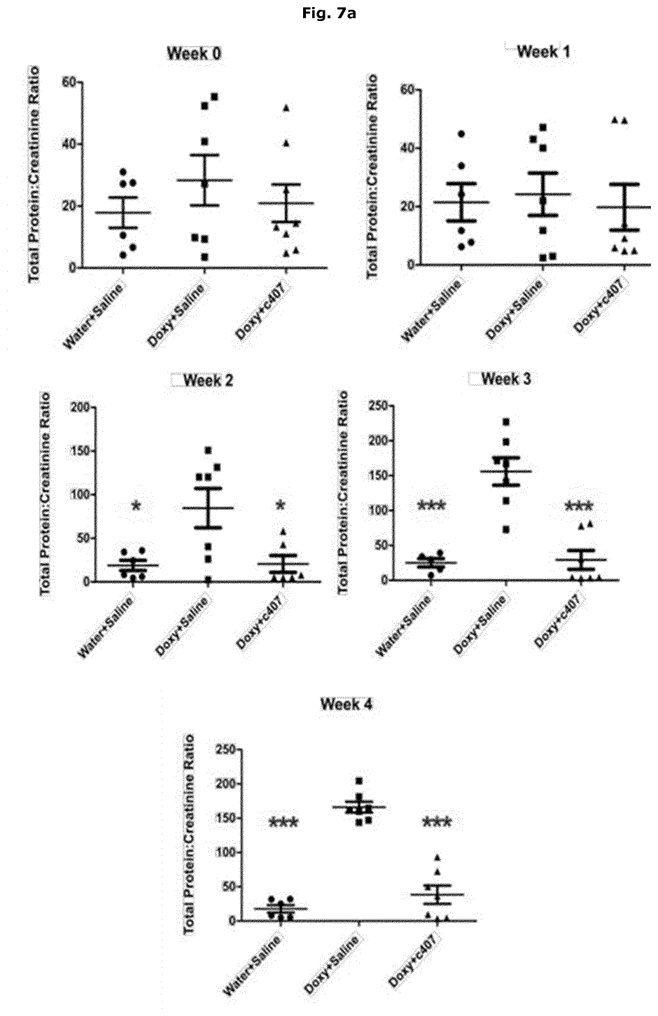

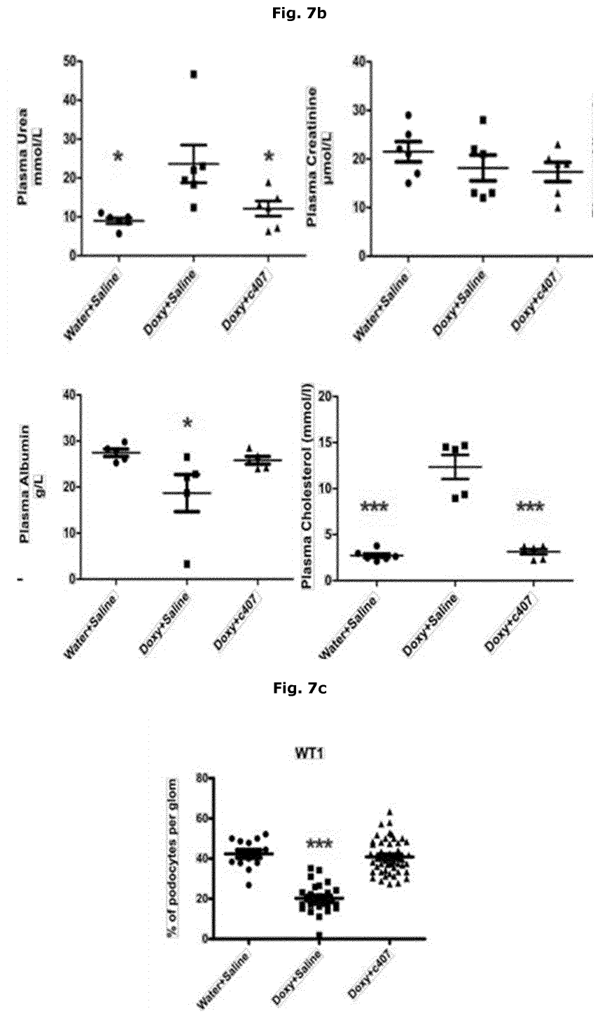

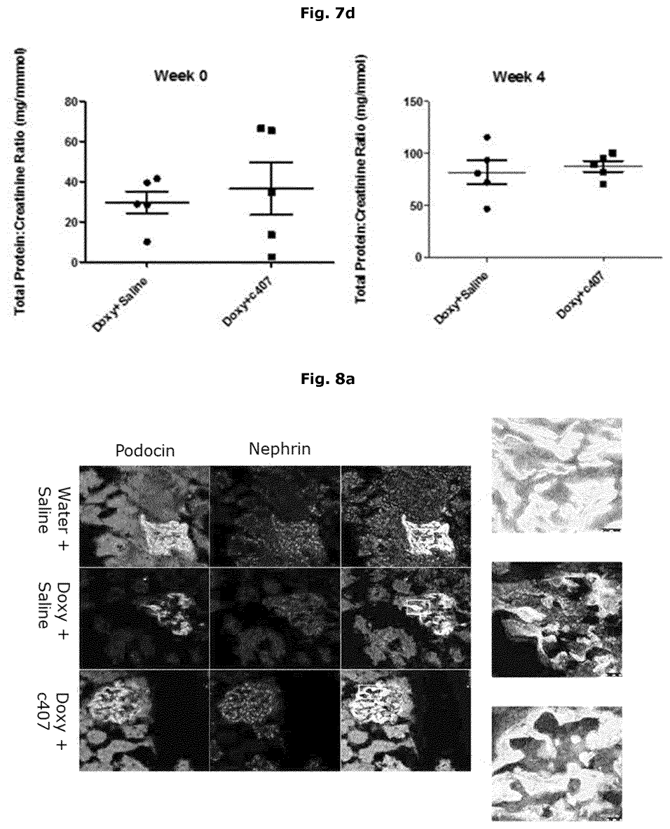

Doxycycline induced animals treated with vehicle, developed proteinuria within the first two weeks of R140Q hemizygosity induction, which peaked at Week 4 as anticipated (FIG. 7a). On the other hand, compound Ia treatment significantly prevented the development of proteinuria in R140Q mice starting from Week 2, and also reversed podocytes loss associated with NS induced by doxycycline (FIG. 7a, c). Furthermore, experimental mice displayed hypercholesterolemia, hypoalbuminemia and high blood urea levels, all of which were prevented upon administration of compound Ia (FIG. 7b). Finally, the Cre/LoxP system was used to excise podocin on both alleles in NPHS2^{flox/flox} mice leading to total podocin knockout in these animals. These mice were used as controls to prove the theory that compound Ia only targets misfolded proteins. These mice displayed no change in proteinuria levels upon administration of compound Ia at disease induction via osmotic mini pumps, as anticipated (FIG. 7d).

### Example 8—Immunofluorescence and Histological Analysis

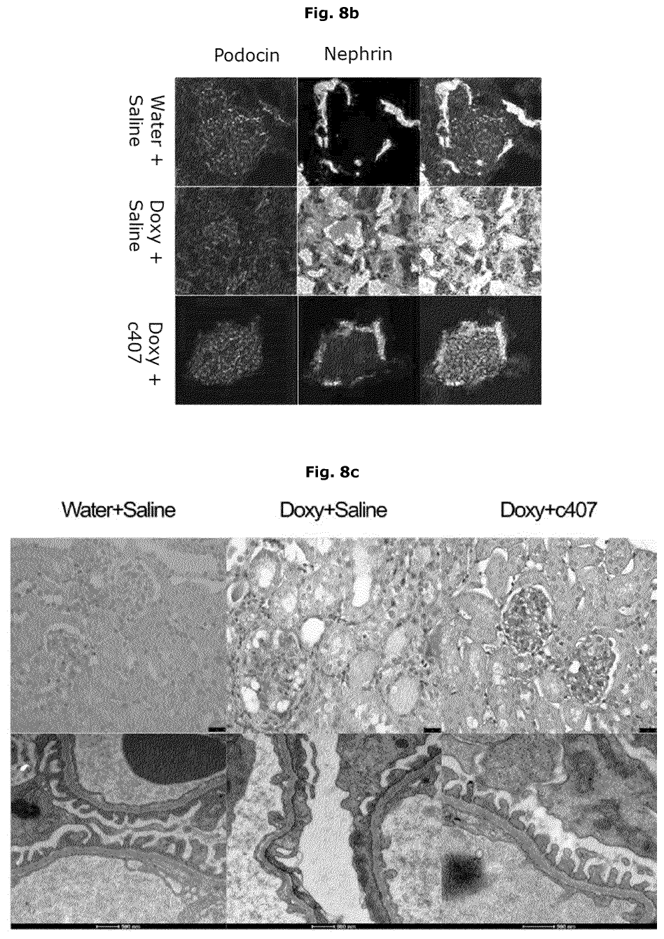

Decreased podocin protein expression was observed at week 4 in in NPHS2^{flox/R140Q} mice, which were given doxycycline for 3 weeks compared to that of healthy controls. In vivo compound Ia treatment restored mutant podocin localization back to the plasma membrane as seen by immunofluorescence on mice kidney sections (FIG. 8a). By contrast, Keratin 8 was seen to be upregulated in glomeruli of mutant mice, and treatment with compound Ia resulted in restoration of minimal keratin 8 expression in glomeruli (FIG. 8b). Furthermore, foot process effacement, FSGS and glomerulosclerosis with protein casts were observed in those animals, as seen on kidney histology and EM, which were prevented with compound Ia treatment (FIG. 8c).

### Example 9—Compound Screening Using Adhesion Assay

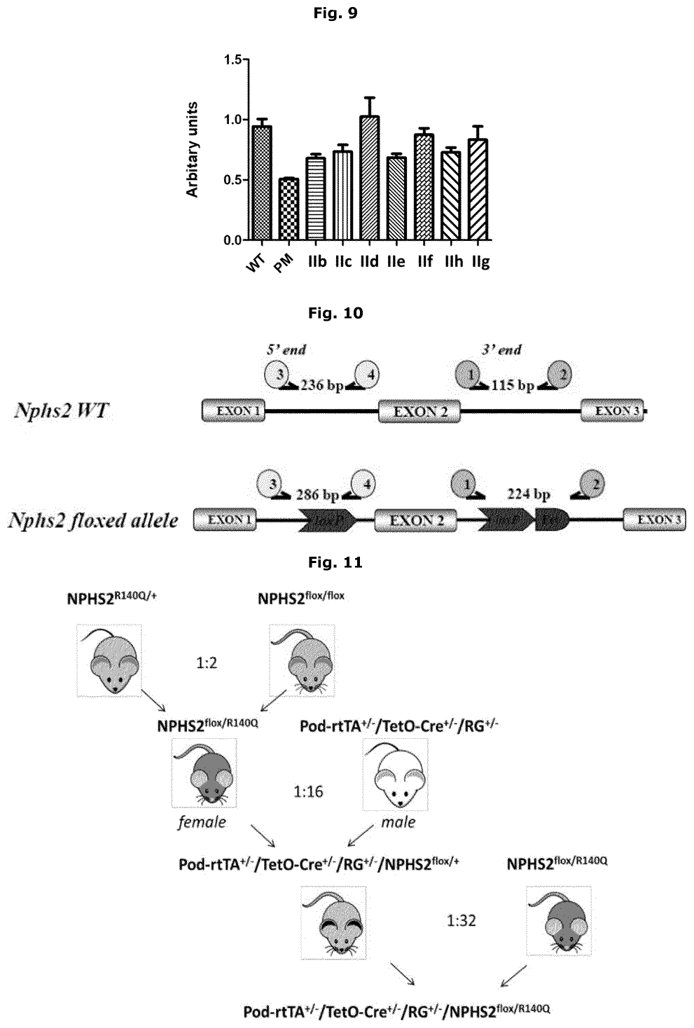

A number of compound Ia analogues have been tested in a robust cell adhesion assay. This is a rapid in vitro assay that provides information on any given compound's ability to restore the WT-phenotype. As can be seen in FIG. 9, all compounds tested provided an improvement.

### Example 10— GlomSphere High-Throughput Assay

It was demonstrated that high throughput screening of inhibitor candidates can be conducted using an InCell analyser with GlomSpheres. GlomSpheres can be used to mimic the glomerulus. They are formed by coating podocytes and endothelial cells with inert nanoparticles, and co-culturing them under magnetic levitation, so that they self organise into a glomerulus-like structure. The use of human podocytes with a podocin mutation can be used to represent the disease condition, and for high-throughput testing of inhibitor candidates. The results of the screening assay conducted using compound 407 are shown in FIG. 12. Specifically, FIG. 12a) shows a podocin R138Q mutant (PM) GlomSphere. Podocytes (green fluorescence) are shown to incompletely cover the GlomSphere surface. FIG. 12b) shows untreated WT (wild type) GlomSphere. Podocytes show much greater coverage of the GlomSphere surface, increasing its apparent size. FIG. 12c) shows a podocin mutant spheroid treated for 5 days with compound 407. Podocyte retention has been improved and the GlomSphere looks more similar to the wild type condition. FIG. 12d) shows quantified GFP mean fluorescence intensity of GlomSpheres made with podocin mutant podocytes, treated with compound 407. The compound can be seen to restore GFP fluorescence (and therefore podocyte retention) to closer to wildtype (WT) levels when compared to untreated GlomSpheres (PM). FIG. 12e) shows the same experiment as FIG. 12d), but with the GFP fluorescence adjusted for GlomSphere size (quantified integrated density of GlomSphere). The assay has demonstrated compound 407's capacity to protect podocyte retention, demonstrating the effectiveness of the assay.

### Example 11— Human Biopsy Immunohistochemistry

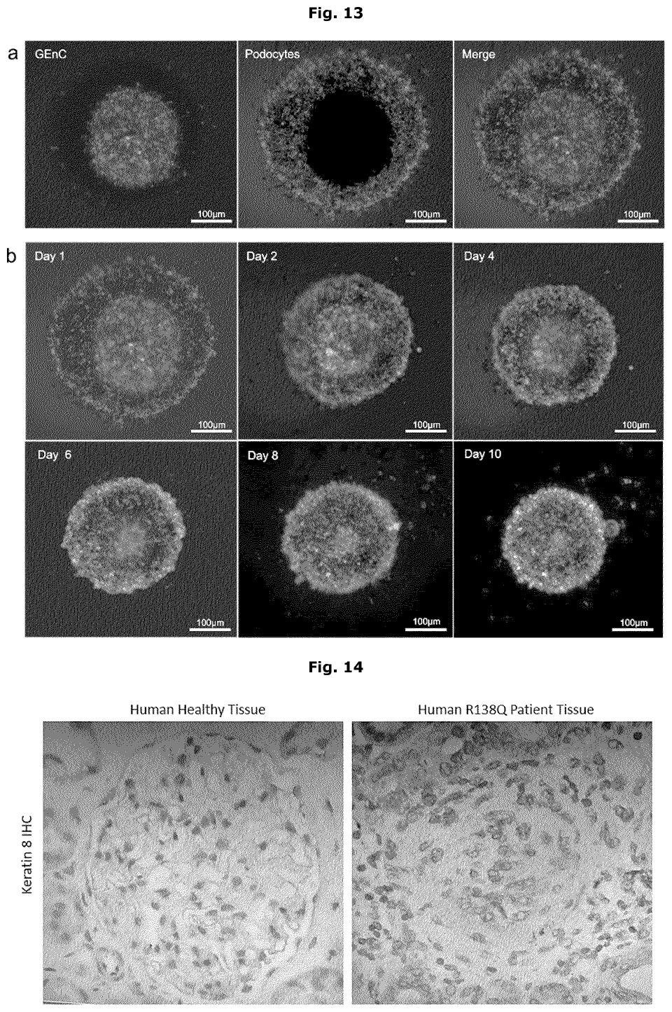

Further evidence of Keratin 8 involvement in removal of R138Q mutated podocin was established through immunohistochemistry performed on human biopsy samples. FIG. 14 shows immunohistochemistry staining for Keratin 8 on human glomerulus biopsies. The left panel shows normal human glomerulus and the right panel shows a biopsy from a patient with a R138Q podocin mutation. Staining for Keratin 8 indicates greatly elevated levels of Keratin 8 in the right panel. Proximal tubular expression is also noted.

### Materials and Methods

All the laboratory reagents were of the highest quality and purity and purchased from Sigma Aldrich unless otherwise specifies in the text or Appendix 1. Appendix 1 also contains list of solutions used, cell culture and cell extraction reagents.

### Cell lines

The wild-type and R138Q podocin mutant conditionally immortalized human podocyte cell lines were developed at Bristol Renal Unit by transduction with the temperature-sensitive SV40-T transgene as previously described (M. A. Saleem et al., J. Am. Soc. Nephroi., vol.

13, no. 3, pp. 630-8, Mar. 2002). These cells can proliferate at the permissive temperature of 33° C., and thereafter they can be transferred to the nonpermissive temperature of 37° C., where the cells enter growth arrest and show key characteristics of podocyte differentiation and function. The wild-type podocyte cell line is referred to as WT, while the podocin mutant podocyte cell line is called PM. The wild-type and R138Q GFP-tagged immortalized human podocyte cell lines were created using PCR-based molecular cloning approach and used in experiments, where stated. All cells were grown and maintained in CO2 incubators with a temperature of 33° C./37° C., 5% CO2 concentration and 95% relative humidity. All cell work was performed in aseptic conditions in a class two biological safety hood. Cell culture media was changed every 34 days.

### Podocytes

All human conditionally immortalised podocyte cell lines were grown in RPMI 1640 media with supplements as detailed in Appendix. Cells were cultured at 33° C. until 70% confluent, and then switched to 37° C. for 10-14 days differentiation. All podocyte cell lines were cultured under sterile conditions in tissue culture vessels including T175 cm², T75 cm² and

T25 cm2 flasks, and 6 well plates. For immunofluorescence cells were grown as described above either on glass coverslips in 6 well plates or in 6 cm2 glass bottom dishes.

### Cell Passage

Stock flasks of proliferating cells were maintained under an atmosphere of 5% (v/v) CO2 and 95% air at the permissive temperature of 33° C. for podocytes. Cells were grown to the desired confluency (70%-90%) and sub-cultured to maintain them at a logarithmic growth rate. For sub-culturing, growth medium was removed, and the adherent cells were washed with 1ml sterile trypsin. The cells were detached from the flask by mild tryptic digestion using 0.5 ml 0.25% Trypsin-EDTA and incubation at 37° C. for 3-5 mins. The cells were then resuspended in the appropriate growth medium and aliquoted into fresh tissue culture flasks or dishes. Each time the cells were split, a consecutive passage number was assigned. Passages 13 to 22 were used for experiments.

### Inhibitor Compounds

The inhibitor compounds were commercially available or synthesized using routine methods known in the art. Compound Ia was a kind gift from Prof. Aleksander Edelman, although it is noted this compound can be synthesized, for example, according to methods disclosed in Acta Crystallogr Sect E Struct Rep Online. 2012 Aug 1; 68(Pt 8): o2456.

Compounds Ib-f were obtained from the vendor ChemBridge, with catalogue numbers as follows: compound Ib=ChemBridge 5116054; compound Ic=ChemBridge 5116157; compound Id=Chembridge 5231281; compound Ie=ChemBridge 5243784; compound If =ChemBridge 5304459; compound Ig=ChemBridge 5304787; compound Ih=ChemBridge 5307860.

### Adhesion Assay

Cell adhesion assays are widely used to assess the adhesion properties of many cell types, for example epithelial cells to the extracellular matrix, other cells, or specially coated surfaces. In addition, this type of assay can be used to determine the effects of various treatments, such as pharmacological compounds and small molecules, on the ability of cells to adhere. An adapted cell adhesion assay protocol is detailed here for studying the adhesion characteristics of human podocytes in vitro. Podocytes were grown in a T75 cm² flask and differentiated at 37° C. for 10-14 days. When fully differentiated, cells were trypsinised with 0.025% trypsin/EDTA for 5 min at 37° C. Podocytes were then resuspended in cell culture media to stop enzyme activity and collected in a 15 ml falcon. Cells were centrifuged for 5 min at 1000 g, and then gently resuspended in 1 ml of cell media. 10-15p1 of cells with trypan blue were pipetted onto the disposable slide and counted with the Luna-FLTM automated cell counter. Podocytes were again resuspended to a concentration of 5×10⁵/ml in cell media. Cells were allowed to recover from trypsinisation in an upright falcon tube with the lid off at 37° C. for 10 min. 50p1 of PBS and 50p1 of cells were added to each well of a 96 well plate. Three experimental wells were assigned as the 100% attachment control, to which 20%, 50% and 100% of the total volume of cells were added. Cells were left to adhere for 45 min at 37° C. Control wells for 100% attachment were fixed with 100 μl 4% PFA for 20 min at room temperature. Thereafter, the plate was tapped to remove lose and non-adherent podocytes, and washed twice with 100 μl PBS, and the experimental wells were then fixed with 100 μl 4% PFA for 20 min (note that the 100% attachment wells were not washed). The PFA was then washed off three times with 100p1 of distilled water, and cells were stained 100p1 0.1% crystal violet in 2% ethanol for 60 minutes at room temperature. Crystal violet was also added to three empty wells in order to measure binding of the dye to the plastic as a control. Crystal violet was removed, and wells were washed 3 times with 400p1 of distilled water. 100p1 of 10% acetic acid was added to each well to solubilise the dye. A 96 well plate was incubated on an orbital shaker at 150 rpm for 5 minutes at room temperature. Absorbance of the plate was measured at 570 nM in a plate reader. Results were expressed as a percentage of 100% attachment and normalised against the adhesion of the human wild type podocytes cell line.

Podocyte adhesion in vitro is widely accepted as a surrogate of podocyte dysregulation in nephrotic syndrome in vivo (ref Welsh GI, Saleem MA. Nat Rev Nephrol. 2011 Oct 25;8(1):14-21). It reflects disruption of cytoskeletal dynamics, and the subsequent podocyte foot process effacement which is common to all forms of human NS.

### GlomSphere High-Throughput Assay

### GlomSphere Cell Culture

Spheroids were formed using a modified version of N3D bioscience standard protocol for magnetic spheroid bioprinting (Nano3D Biosciences Inc). A T75 flask of cells (i.e. endothelial cells or podocytes) was incubated overnight with 100p1 nanoshuttle-PL (Nano3D Biosciences Inc), added to 10 ml of fresh cell culture medium. Cells were then washed with 5 ml sterile phosphate-buffered saline (PBS) and with trypsin-EDTA (Lonza). Cells were then pelleted via 1500 rpm centrifugation (5 mins) and counted with a Luna cell counter (Logos Biosystems). For monoculture experiments, 10,000 cells were pipetted into each well of an ultra-low attachment plate (Greiner) and a 96-magnet MagDrive (Nano3D Biosciences) was placed underneath. Spheroids were left to form overnight at 33° C.

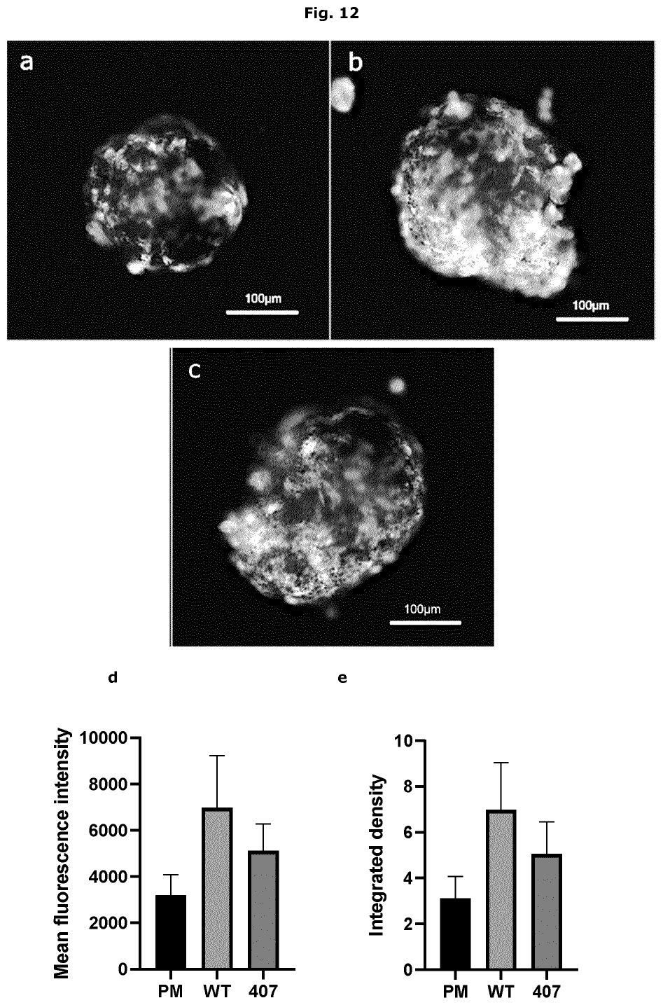

For co-culture/GlomSphere spheroids, a spheroid of 5000 glomerular endothelial cells (GEnCs) was generated as above and allowed to aggregate for 1 hr at 33° C. The MagDrive was then removed and 5000 podocytes were pipetted into each well before the MagDrive was replaced. This forces the newly added podocytes to form a peripheral coating around the GEnC core. The spheroids were then thermoswitched to 37° C. and the podocyte layer migrates (FIG. 13). After 10 days of differentiation, treatment or fixation was performed.

### Whole-Mount Fixation and Immunofluorescent Staining

GlomSpheres were magnetically transferred to Eppendorf tubes and fixed in 4% Paraformaldehyde (Sigma) containing 1% Triton-x100 (Sigma) (20 mins, 20° C.). To block, spheroids were incubated with 5% bovine-serum-albumin (BSA) (Sigma) containing 0.1% Triton-x100 (overnight, 4° C.). Spheroids were then incubated with primary antibody, diluted in 5% BSA containing 0.1% Triton-x100 (48 hours, 4° C.). Spheroids were then washed in PBS containing 1% Triton-x100 (3x30 minutes, room temp). Spheroids were then incubated with secondary fluorescent antibodies diluted 1:400 in 5% donkey serum (Sigma) containing 0.1% Triton-x100 (24 hours, 4° C.). Spheroids were then washed in PBS containing 1% Triton-x100 (3x30 minutes, 20° C.).

### Sectioning and 2D Immunofluorescent Staining

Sections were cut as previously described (Tuffin J, Burke M, Richardson T, Johnson T, Saleem MA, Satchel) S, et al. A Composite Hydrogel Scaffold Permits Self-Organization and Matrix Deposition by Cocultured Human Glomerular Cells. Adv Healthc Mater. 2019;8(17):e1900698). Sections or fixed 2D-cultured cells on glass coverslips were washed in PBS (2x 5 mins, 20° C.). To block, samples were incubated in 5% BSA containing 0.1% Triton-x100 (45 minutes, 20° C.), then incubated with primary antibody, diluted in 5% BSA containing 0.1% Triton-x100 (1 hour, 20° C.). Primary antibodies were then washed with PBS (2x 5 mins, 20° C.) before being incubated with fluorescent secondary antibodies diluted in 1:400 in 5% donkey serum (1 hour, 20° C.). Samples were then washed with PBS (2x 5 mins, 20° C.) and mounted to microscope slides with 5p1 Mowiol (Sigma).

### High Throughput Screen

Spheroids were formed using either GFP CI podocytes (WT) or GFP CI podocin mutant podocytes (PM). Compounds were added 24 hrs after culture and re-dosed daily. Images were taken daily and those shown and quantified are from Day 5. Imaging was performed using an IN cell analyser (GE lifesciences).

### Mouse model of R140Q podocin mutant

To test whether inhibitor compounds can rescue mutant podocin localization in vivo, a transgenic mouse model was developed and characterized. This mouse was designed to carry the R140Q mutation, the mouse analogue of human R138Q, on one allele and floxed WT NPHS2 on the other, which can be excised upon induction with doxycycline.

A mouse model carrying R140Q podocin mutation, which is analogous to human p.R138Q variant, was generated using a 6.6 kb targeting construct (A. Philippe et al., Kidney Int., vol. 73, no. 9, pp. 1038-47, May 2008). Using site-directed mutagenesis, the c.505G>A, c.506A>G mutations were introduced into exon 3 of the NPHS2 gene of the targeting vector, while a phosphoglycerate kinase— hygromycin cassette flanked by flox sites was inserted into intron 3 to select positive embryonic stem (ES) cell clones. Following successful homologous recombination, two ES cell clones were selected and injected into the murine blastocyst of C57BL/6 mice. Several rounds of breeding were undertaken to generate a mouse with germline incorporation of the mutant allele, which was further mated with mice constitutively expressing Cre recombinase that aids the removal of the floxed hygromycin insert. Using this model, the group of C. Antignac have demonstrated that homozygous NPHS2 R140Q/R140Q mice die within the first week of life with some mice dying during embryogenesis, while heterozygous NPHS2^{R140Q/+}mice did not develop albuminuria or any renal anomalies. The above models are characterized either by severe renal phenotype resulting in early death or by insufficient expression of the mutated protein. Therefore, the conditional inactivation of wild-type protein is essential to elucidate the role of podocin in health and disease in mice at postnatal age.

In more detail, homozygous NPHS2^{flox/flox} and heterozygous NPHS2^{R140Q/+}transgenic mice on a 129Sv/PasCrl genetic background were a kind gift from Prof. Corinne Antignac (INSERM, Paris). Homozygous NPHS2^{flox/flox} mice were generated to carry a floxed NPHS2 exon 2 alleles, which can be excised, when Cre recombinase is expressed leading to a conditional podocin inactivation in mature kidneys (FIG. 10) (G. Mollet et al., J. Am. Soc. Nephrol., vol. 20, no. 10, pp. 2181-9, Oct. 2009.). Initially, two lines NPHS2^{R140/+}and NPHS2R^{R140Q/+}were crossed to generate heterozygous NPHS2^{flox/R140Q}offspring mice, which were used for further breeding (FIG. 11). Subsequently, the NPHS2 ^{flox/R140Q} mice were crossed with a transgenic iPod male mouse model of a mixed genetic background (a kind gift from Prof. Richard Coward) for generation of the inducible mice line. The iPod male mouse model is composed of three genes: 1) rtTA that is under the control of a 2.5 kb NPHS2 promoter driving podocyte-specific expression of rtTA (T. Shigehara et al., J. Am. Soc. Nephrol., vol. 14, no. 8, pp. 1998-2003, Aug. 2003.); 2) TetO-Cre that is under the control of a tetO, to which the rtTA protein binds in the presence of tetracycline or analog, such as doxycycline, thus allowing Cre recombinase expression; 3) RG reporter, also known as mT/mG, that is a red green fluorescent reporter under the control of a CMV enhancer/chicken beta actin core promoter that regulates the expression of a loxP-flanked tdTomato reporter element followed by a polyadenylation signal and EGFP reporter element. Mouse podocyte cells initially express red fluorescence, and then green fluorescence in the presence of Cre recombinase, which can excise floxed tdTomato reporter, suggesting that the R140Q allele is in the heterozygous state. Succeeding multiple rounds of breeding, the resulting transgenic mice had the specified cassette of genes (Pod-rtTA⁺/⁻TetO-Cre⁺/⁻NPHS2^{flox/R140Q}RG⁺/⁻or Pod-rtTA⁺/⁻TetO-Cre⁺/⁻NPHS2^{flox/R140Q}RG⁻/⁻) and were used for the final experiments. For simplicity, the mice are called NPHS2^{flox/R140Q} in this document.

### Experimental Design for Phenotypic Characterization

Postnatal induction of R140Q hemizygosity in NPHS2^{flox/R14Q} mice was achieved by administration of doxycycline at 2 mg/ml in 5% sucrose dissolved in drinking water for three weeks. Doxycycline, a tetracycline derivative, binds with high affinity to rtTA, thus inducing a Tet-off system in transgenic mice. Doxycycline was shown to have an excellent medical safety record and deep tissue penetration with relatively low toxicity in eukaryotic cells (I. M. Redelsperger et al., J. Am. Assoc. Lab. Anim. Sci., vol. 55, no. 4, pp. 467-74, 2016.). Doxycycline was made fresh every 4 days and administered in black bottles to protect from light. Control mice with the same genotype (NPHS2^{flox/R140Q}) were given the same volume of drinking water without doxycycline. Other control groups of mice, which lacked either Pod-rtTA or TetO-Cre or both, received the equivalent volume of drinking water with doxycycline at the same concentration as used for the experimental mice. The rationale for having mice without the Pod-rtTA gene, for example, is that the induction efficiency of the floxed wild-type podocin can be monitored. Mice were maintained and managed by skilled animal technicians in the pathogen-free conditions of ASU unit, University of Bristol.

Beginning from the first week after doxycycline induction, weekly urine collections were performed to look for the onset and control the duration of proteinuria until the point of sacrificing. Animals were sacrificed at weeks 10-12 after doxycycline administration. In case of weight loss (over 10%), the affected animal was culled immediately. At designated experimental time points mice were maintained under general anaesthesia with isoflurane, and 1 ml of blood was withdrawn by cardiac puncture. Animals were subsequently sacrificed by Schedule 1, and both kidneys were removed (flash frozen, put in PFA or EM buffer).

Samples were collected and used for RNA and protein extraction, immunofluorescence studies, immunohistochemistry and electron microscopy. Urine albumin concentration was analysed using human albumin ELISA kit (Bethyl Laboratories). Whole blood samples were sent to Diagnostic Laboratories (Langford Vets, University of Bristol) to measure serum creatinine, albumin and urea. Urine samples were also sent to Diagnostic Laboratories (Langford Vets) to analyse creatinine in the urine.

### NPHS2^{flox/R140Q} Mice Develop NS and End-Stage Kidney Disease

Doxycycline induction of a hemizygous state in the NPHS2^{flox/R140Q} mice led to severe proteinuria detectable within few days, which peaked to a maximum at 2-4 weeks after induction of NPHS2^{nU!!/R140Q} and was maintained at week 12 (FIG. 4.4a). Consequently, there was a continuous decrease in proteinuria observed from week 6 in experimental animals, but it still stayed elevated compared to controls until the point of sacrificing at 12 weeks. Furthermore, proteinuric animals had a significantly reduced weight gain from week 8 onward, when compared to healthy controls (FIG. 4.4c). The NPHS2nU!!R140Q animals presented with hypoalbuminemia (g/L), hypercholesterolemia (mmol/L), uremia (mmol/L) and abnormally high plasma creatinine levels (pmol/L) at the point of sacrifice.

### Drug Delivery Via ALZET Osmotic Pumps

Pod-rtTA^{+/-}TetO-Cre^{+/-}NPHS2^{flox/R140Q}RG^{+/-or Pod-rtTA}^{^{+/−TetO-Cre}}^{+/-}NPHS2^{flox/R140Q}RG^{-/-}transgenic mice (NPHS2^{flox/R140Q} mice for simplicity) were used to see whether prolonged treatment with compound Ia corrected the altered podocin localization and prevented proteinuria in these mice. ALZET Osmotic Pumps 2004 were chosen to administrate compound Ia/0.9% NaCl at a continuous and controlled rate for 28 days. Subcutaneous pump implantation was performed to deliver compound Ia dissolved in 0.9% NaCl or 0.9% NaCl for 28 days as previously described (K. Di Gregoli et al., Circ. Res., vol. 120, no. 1, pp. 49-65, Jan. 2017). Mice were anaesthetized with 3% isoflurane (with oxygen at 1 L/min), and the same level of anaesthesia was maintained during osmotic pump implantation.

Directly after surgery, subcutaneous injection of rimadyl (0.1 mg/kg) was administrated as analgesia. Mice were closely monitored for 24 h and expected to make a rapid recovery from implantation of osmotic pumps. Wound clips were removed at day 11 post-surgery. ALZET Osmotic pumps are designed to have a fixed volume-delivery rate, therefore accurate calculations based on the volume of impermeable pump reservoir were performed to fill each pump with appropriate volume of inhibitor to achieve a dose of 22.3 mg/month/mouse. ALZET Osmotic Pumps 2004 operate based on the difference in an osmotic pressure between the osmotic layer of the pump and the subcutaneous tissue layer of the living animal. The high osmolarity of the osmotic layer results in water being diffused into the pump through a semipermeable membrane creating an increase in pressure within the pump, which in turn displaces the contents from the pump at a controlled rate. This further results in the impermeable reservoir of the pump being compressed; thus, the pump is designed for a single use only.

### Urine Collection

Urine samples were obtained from all mice prior to the start of any experimental procedures, such as doxycycline induction or implantation of the osmotic pumps. Beginning from the first week after doxycycline induction, weekly urine collections were performed to look for the onset and control the duration of proteinuria until the point of sacrificing (10-12 weeks)in NPHS2^{flox/R140Q} mice. In the compound Ia/saline osmotic pump model, urine was collected twice a week for a 28-day period. To collect samples, mice were placed individually in a clean plastic container and watched constantly. Once the mouse urinated, 50-100 μl of urine was pipetted off and stored at −20° C. until analysis. Siemens Multistix Urinalysis strips were used as a basic diagnostic tool to give an estimation of proteinuria as indicated by a colour change on the protein pad.

### Kidney Tissue

The kidneys harvested from the NPHS2^{flOx/R140Q} mice were processed in several ways to provide a phenotypic profile for each animal after the transgene induction and to examine the effects of compound Ia on the renal function.

Mice were culled in accordance with Schedule 1 humane killing methods by confirmation of permanent cessation of the circulation. Both kidneys were collected and cut in half longitudinally. Half of one kidney was fixed in 10% (v/v) formalin in PBS for four days, before being placed in 70% ethanol. Two other halves were placed in cryovials and snap frozen immediately in liquid nitrogen. The cryovials were then stored at −80° C. until required for IF, RNA and protein extraction. The last half of the kidney was cut into 1 mm pieces and placed in the EM buffer for fixation and storage at 4° C. Spleen was also harvested and snap frozen immediately from each animal to be used as a control tissue for RNA and protein.

Kidney tissues were fixed in 10% (v/v) formalin, further processed and paraffin embedded after ethanol dehydration. Tissues were then cut by University of Bristol histopathology staff within the Bristol Medical School histopathology laboratory to give sections 3 μm thick.

### Tissue Staining -Periodic Acid-Schiff (PAS) Staining

PAS staining was performed on paraffin-embedded tissue sections to evaluate the degree of sclerosis within the glomerular tuft using PAS staining system (SIGMA-ALDRICH) as per manufacturer's protocol. Slides were immersed twice in Histo-Clear II (National Diagnostics HS-202) for 5 min to deparaffinize sections. Sections were then rehydrated by passing the slides through alcohol series (100% ethanol, 90% ethanol, 70% ethanol, 50% ethanol) to deionized water. Slides were oxidized in periodic acid solution for 5 min at room temperature, before being rinsed in several changes of distilled water. Slides were then placed in Schiff's reagent for 15 min and washed in running tap water for 5 min. The tissue sections were counterstained in hematoxylin solution for 90 seconds and immediately washed again in running tap water. The sections were then dehydrated by being passed through alcohol series (50% ethanol, 70% ethanol, 90% ethanol, 100% ethanol), dried and mounted in DPX (Sigma #44581). The sections were then imaged using a Leica light microscope that allows to assess morphology of the kidney.

### Tissue Staining -Masson's Trichrome Staining

Masson's Trichrome staining was performed on paraffin-embedded tissue sections to evaluate the degree of glomerular fibrosis (collagen deposits) using Trichrome Stain

(Masson) Kit ((SIGMA-ALDRICH) as per manufacturer's protocol. Slides were immersed twice in xylene for 5 min to deparaffinize sections. Sections were then rehydrated by passing the slides through alcohol series (100% ethanol, 90% ethanol, 70% ethanol, 50% ethanol) to deionized water. Slides were stained in Weigert's iron hematoxylin working solution for 5-10 mins and then rinsed in running warm tap water for another 10 mins, followed by the distilled water wash. The tissue sections were then stained in Biebrich scarlet-acid fuchsin solution for 10-15 mins, washed in distilled water and differentiated in phosphomolybdic-phosphotungstic acid solution for another 5-10 mins (or until collagen is not red). Sections were then transferred directly to aniline blue solution and stained for 5-10 mins, followed by a brief rinse in distilled water and differentiation in 1% acetic acid solution for 2-5 mins, followed by a brief wash in distilled water. The sections were then dehydrated by being passed through alcohol series and cleared in xylene, dried and mounted in DPX (Sigma #44581). The sections were then imaged using a Leica light microscope that allows to assess morphology of the kidney.

### Immunohistochemistry (IHC)

IHC was performed with Wilm's tumour-1 protein (WT1) antibody (Santa Cruz) to look for the glomerular podocyte number in mice kidney sections. Slides were immersed twice in histoclear for 5 min to deparaffinize sections. Sections were then rehydrated by passing the slides through alcohol series (100% ethanol, 90% ethanol, 70% ethanol, 50% ethanol) to deionized water. Slides were next boiled in the antigen retrieval buffer (10 mM Sodium citrate tribasic, pH 6) for 10 min in microwave and allowed to cool on bench top for 30 min.

Sections were then washed in deionized water 3 times for 5 min each. PAP pen was used to draw around sections, which were then incubated in 3% hydrogen peroxide for 20 min in a humidified box, followed by wash in deionized water twice for 5 min each. Sections were blocked in blocking solution (5% goat serum in TBST 0.1%) for 30 min at room temperature. Blocking solution was then removed, and sections were incubated with primary antibody (20u1/sample) overnight at 4° C. Next morning, primary antibody was removed, and sections were washed with wash buffer (TBST 0.1%) 3 times for 5 min each. Sections were then covered with 1-3 drops of SignalStain® Boost Detection Reagent and incubated in a humidified chamber for 30 min at room temperature. Meanwhile, 1 drop (30 p1) SignalStain® DAB Chromogen Concentrate was added to 1 ml SignalStain® DAB Diluent and mixed well before use (20u1/sample). Following 3 washes with wash buffer, SignalStain® DAB was applied to each section for 1 to 10 mins, or until the brown staining became apparent. Slides were then immersed in deionized water and counterstained with hematoxylin for 10 seconds. Next, slides were washed in tap water, until water became clear. The sections were then dehydrated by being passed through alcohol series and cleared in histoclear, dried and mounted in DPX (Sigma #44581). The sections were then imaged using a Leica light microscope.

### Electron Microscopy (EM)

Kidney tissue preparation for immersion fixation involved rapid excision of animal tissues and immersion of small tissue pieces (1 mm cubes) in fixative (2.5% glutaraldehyde in 0.1 M cacodylate at pH 7.3) in order to get the best ultrastructure. Tissues were then washed in 0.1 M cacodylate buffer 3 times for 10 minutes, followed by post fixation in 1% osmium tetroxide in 0.1M cacodylate buffer pH 7.3 at 4-8° C. for 1 hour. Kidney tissues were washed in 0.1 M cacodylate buffer for 15 minutes, followed by 3 washes in deionised water. Tissues were then put into 1-3% Uranyl acetate en bloc staining overnight, followed by 15-minute deionised water wash next morning. Kidney tissues were washed twice in propylene oxide for 15 minutes and infiltrated with Epon resin mix: propylene oxide (1:1) for 2 hours. Infiltrated tissues were then embedded in fresh Epon resin mix in a silicon rubber mould and left to polymerize for >24 hours at 60° C. Note, Epon resin mix consists of resin, hardener, accelerator and plasticiser. Finally, tissues were cut into sections (70 nm thick) on an Ultracut S ultramicrotome, which were then stained with 3% uranyl acetate (5 mins) and Reynolds' lead citrate (2 mins) with deionised water wash in between and after the lead stain. Sections of glomerulus were imaged at various magnifications in a Tecnai T12 microscope (FEI Ltd).

1. A compound for use in the treatment of nephrotic syndrome in a subject in need thereof, wherein the compound is an inhibitor of binding between a target protein and keratin 8.

2. A compound for use according to claim 1, wherein the target protein is podocin.

3. A compound for use according to claim 1 or claim 2, wherein the nephrotic syndrome is steroid resistant nephrotic syndrome (SRNS).

4. A compound for use according to any preceding claim, wherein the compound comprises at least one moiety selected from phosphinic acid moieties, phosphinate ester moieties and pharmaceutically acceptable salts thereof.

5. A compound for use according to any preceding claim, wherein the compound is a compound of formula I, or a pharmaceutically acceptable salt thereof: 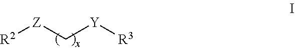
wherein x is 0 or an integer from 1 to 6;
Y is selected from the group consisting of: direct bond, C(0), C(O)O, O, C(R¹)(OH), C(O)NR¹, S(O), S(O)(O) and P(O)(OR¹);
Z is selected from the group consisting of: C(O), C(O)O, O, C(R′)(OH), C(O)NR¹, S(O), S(O)(O) and P(O)(OR′);
wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;
R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl; and
R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

6. A compound for use according to any preceding claim, wherein the compound is a compound of formula II, or a pharmaceutically acceptable salt thereof: 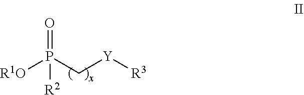
wherein R¹ is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;
R² is selected from the group consisting of: hydrogen, optionally substituted alkyl, optionally substituted cycloalkyl, optionally substituted alkenyl, optionally substituted alkynyl, optionally substituted aryl, optionally substituted heteroaryl and optionally substituted heterocyclyl;
x is 0 or an integer from 1 to 6;
Y is selected from the group consisting of: direct bond, C(O), C(O)O, C(O)NH, S(O), and P(O)(OH); and
R³ is selected from the group consisting of: hydrogen, OH, optionally substituted alkyl, optionally substituted cycloalkyl, optionally substituted alkenyl, optionally substituted alkynyl, optionally substituted aryl, optionally substituted heteroaryl, optionally substituted heterocyclyl, optionally substituted alkoxy, COOH, optionally substituted carboxylate ester, optionally substituted amide, optionally substituted amine, optionally substituted ether, phosphinic acid and phosphinate ester.

7. A compound for use according to claim 6, wherein:
R¹ is selected from hydrogen, optionally substituted C₁₋₁₂ alkyl and optionally substituted C₃₋₁₂ cycloalkyl;
R² is selected from optionally substituted C₆₋₁₂ aryl and optionally substituted C₅₋₁₂ heteroaryl;
Y is selected from the group consisting of: direct bond, C(O), S(O), C(O)O, C(O)NH, and P(O)(OH); and
R³ is selected from the group consisting of: hydrogen, optionally substituted C₁₋₁₂ alkyl, optionally substituted C₃₋₁₂ cycloalkyl, optionally substituted C₆₋₁₂ aryl, optionally substituted C₅₋₁₂ heteroaryl, COOH, optionally substituted carboxylate ester, phosphinic acid and phosphinate ester;
wherein the optional substituents are selected from halo, hydroxy, C₁₋₁₂ alkyl, C₁₋₁₂ alkoxy, COOH, carboxylate ester, nitrile, nitro and amine.

8. A compound for use according to claim 7, wherein:
R¹ is selected from hydrogen and C₁₋₆ alkyl;
x is an integer from 1 to 3;
Y is selected from the group consisting of: P(O)(OH); and
R³ is selected from the group consisting of: hydrogen, C₁₋₆ alkyl, optionally substituted phenyl, COOH, and phosphinic acid;
wherein the optional substituents are selected from halo, hydroxy, C₁₋₁₂ alkyl, C₁₋₁₂ alkoxy, COOH, carboxylate ester, nitrile, nitro and amine.

9. A compound for use according to any preceding claim, wherein the compound is selected from compounds of formulae IIa-IIh or a pharmaceutically acceptable salt thereof:

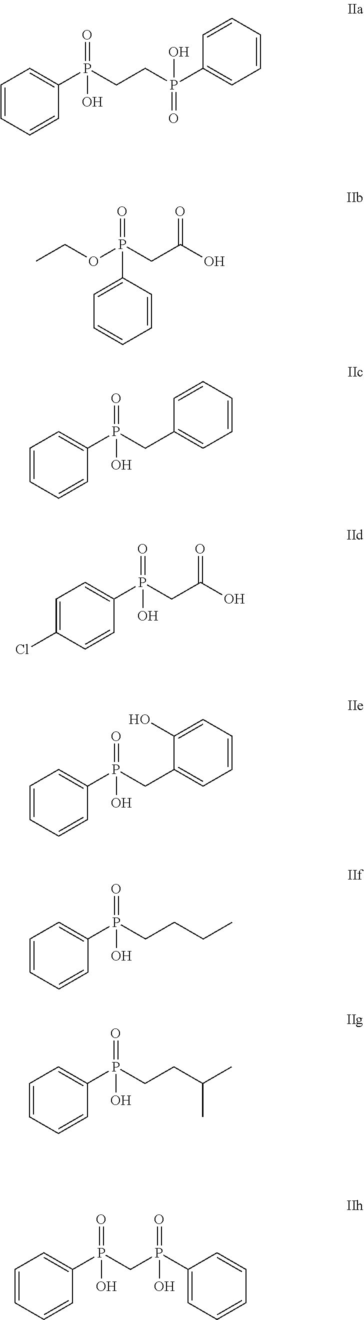

10. A compound for use according to claim 9, wherein the compound has the formula IIa.

11. A kit comprising a compound which is an inhibitor of binding between a target protein and keratin 8 together with instructions for treating nephrotic syndrome.

12. A kit according to claim 11, wherein the target protein is podocin.

13. A kit according to claim 11 or claim 12, wherein the nephrotic syndrome is steroid resistant nephrotic syndrome (SRNS).

14. A kit according to any one of claims 11 to 13, wherein the compound is as defined in any one of claims 4 to 10.

15. A method of treating nephrotic syndrome in a subject in need thereof comprising administering to said subject an effective amount of a compound which is an inhibitor of binding between a target protein and keratin 8.

16. A method according to claim 15, wherein the target protein is podocin.

17. A method according to claim 15 or claim 16, wherein the nephrotic syndrome is steroid resistant nephrotic syndrome (SRNS).

18. A method according to any one of claims 15 to 17, wherein the compound is as defined in any one of claims 4 to 10.

19. The use of a compound which is an inhibitor of binding between a target protein and keratin 8 in the manufacture of a medicament for the treatment of nephrotic syndrome in a subject in need thereof.

20. The use according to claim 19, wherein the target protein is podocin.

21. The use according to claim 19 or claim 20, wherein the nephrotic syndrome is steroid resistant nephrotic syndrome (SRNS).

22. The use according to any one of claims 19 to 21, wherein the compound is as defined in any one of claims 4 to 10.

23. A compound for use according to any one of claims 1 to 10, the method according to any one of claims 15 to 17 or the use according to any one of claims 19 to 22, wherein the subject is a human.
